(19) United States
(12) Patent Application Publication
(10) Pub. No.: US 2019/0365628 A1
(43) Pub. Date: Dec. 5, 2019

(54) COMPOSITION IN THE FORM OF NANO- OR MICRO- EMULSION

(71) Applicant: L'OREAL, Paris (FR)

(72) Inventors: Maki KOIDE, Kawasaki-shi (JP); Kazuko JIMBO, Kawasaki-shi (JP); Sébastien GREGOIRE, Aulnay-sous-Bois (FR)

(21) Appl. No.: 16/348,177

(22) PCT Filed: Nov. 21, 2017
(86) PCT No.: PCT/JP2017/042471

§ 371 Date: May 8, 2019

(30) Foreign Application Priority Data
Nov. 28, 2016 (JP) 2016-229836

(51) Int. Cl. A61K 8/68; A61K 8/34; A61K 8/31; A61K 8/37; A61K 8/39; A61K 8/67; A61K 8/86; A61K 8/06; A61Q 19/10; A61Q 5/02

(52) U.S. Cl. CPC A61K 8/68; A61K 8/342; A61K 8/31; A61K 8/37; A61K 8/39; A61K 2800/10; A61K 8/86; A61K 8/068; A61Q 19/10; A61Q 5/02; A61K 8/675

(57) ABSTRACT

The present invention relates to a composition in the form of a nano- or micro-emulsion, comprising: (a) at least one oil; (b) at least one nonionic surfactant with an HLB value of from 8.0 to 14.0, preferably from 9.0 to 13.5, and more preferably from 10.0 to 13.0; (c) at least one ceramide compound; (d) at least one compound selected from Vitamin B3 and derivatives thereof; and (e) water. The composition according to the present invention can be used as a cosmetic composition and can enhance or improve the penetration of a ceramide compound into a keratin substance such as the skin.

### TECHNICAL FIELD

The present invention relates to a composition in the form of a nano- or micro-emulsion, preferably a cosmetic composition in the form of a nano- or micro-emulsion, including a combination of specific compounds.

### BACKGROUND ART

Ceramide, which is present in the horny layer of the skin, forms a lipid barrier necessary for retaining moisture and plays an important role in the maintaining of moisture in the skin. Ceramide in the horny layer is produced by the breakdown of cerebroside by an enzyme known as cerebrosidase. The ceramide is partially transformed into phytosphingosine and sphingosine by an enzyme known as ceramidase. Phytosphingosine and sphingosine play an important role in the control of cell growth and differentiation. Six different types of ceramides possessing different functions are present in the human skin.

On the other hand, oil-in-water (O/W) or water-in-oil (W/O) emulsions are well known in the field of cosmetics and dermatology, in particular for the preparation of cosmetic products, such as milks, creams, tonics, serums or toilet waters.

In particular, a fine emulsion such as an O/W nano- or micro-emulsion is particularly interesting in cosmetic products due to its transparent or slightly translucent aspect.

For example, WO 2015/152420 discloses a fine emulsion which comprises a ceramide compound. The ceramide compound may be present in dispersed oil phases in the fine emulsion. It is difficult for ceramides to penetrate into a keratin substance such as the skin.

The dispersed oil phase may function as a carrier of the ceramide compound(s) and accelerate the penetration of the ceramide compound(s) into the skin.

### DISCLOSURE OF INVENTION

However, there has still been a need for a composition including ceramide which can further enhance or improve the penetration of the ceramide into a keratin substance such as the skin.

An objective of the present invention is to provide a composition in the form of a nano- or micro-emulsion with a transparent or slightly translucent, preferably transparent, aspect of the emulsion, which can further enhance or improve the penetration of a ceramide compound into a keratin substance such as the skin.

The above objective of the present invention can be achieved by a composition in the form of a nano- or micro-emulsion, comprising:
(a) at least one oil;
(b) at least one nonionic surfactant with an HLB value of from 8.0 to 14.0, preferably from 9.0 to 13.5, and more preferably from 10.0 to 13.0;
(c) at least one ceramide compound;
(d) at least one compound selected from Vitamin B3 and derivatives thereof; and
(e) water.

The (a) oil may be chosen from ester oils, hydrocarbon oils, fatty alcohols and mixtures thereof.

The amount of the (a) oil may range from 0.1 to 30% by weight, preferably from 0.5 to 20% by weight, and more preferably from 1 to 10% by weight, relative to the total weight of the composition.

The (b) nonionic surfactant may be chosen from:
surfactants that are fluid at a temperature of less than or equal to 45° C., chosen from the esters of at least one polyol chosen from the group formed by polyethylene glycol comprising from 1 to 60 ethylene oxide units, sorbitan, glycerol comprising from 2 to 30 ethylene oxide units, polyglycerols comprising from 2 to 12 glycerol units, and of at least one fatty acid comprising at least one saturated or unsaturated, linear or branched C₈-C₂₂ alkyl chain,
mixed esters of fatty acid or of fatty alcohol, of carboxylic acid and of glycerol,
fatty acid esters of sugars and fatty alcohol ethers of sugars,
surfactants that are solid at a temperature of less than or equal to 45° C., chosen from fatty esters of glycerol, fatty esters of sorbitan and oxyethylenated fatty esters of sorbitan, ethoxylated fatty ethers and ethoxylated fatty esters,
block copolymers of ethylene oxide and of propylene oxide,
polyoxyethylenated (1-40 EO) and polyoxypropylenated (1-30 PO) alkyl (C₁₆-C₃₀) ethers, and
silicone surfactants.

It may be preferable that the (b) nonionic surfactant be chosen from:
polyethylene glycol isostearate or oleate (8 to 10 mol of ethylene oxide),
polyethylene glycol isocetyl, behenyl ether or isostearyl ether (8 to 10 mol of ethylene oxide),
polyglyceryl monolaurate or dilaurate comprising 3 to 6 glycerol units,
polyglyceryl mono(iso)stearate comprising 3 to 6 glycerol units,
polyglyceryl monooleate comprising 3 to 6 glycerol units, and
polyglyceryl dioleate comprising 3 to 6 glycerol units.

The (b) nonionic surfactant may be chosen from polyglyceryl fatty acid esters, preferably esters of a fatty acid and polyglycerine comprising 70% or more of polyglycerine whose polymerization degree is 4 or more, preferably esters of a fatty acid and polyglycerine containing equal to or more than 60% of polyglycerine whose polymerization degree is between 4 and 11, and more preferably esters of a fatty acid and polyglycerine containing equal to or more than 30% of polyglycerine whose polymerization degree is 5.

The amount of the (b) nonionic surfactant may range from 0.1 to 30% by weight, preferably from 0.5 to 20% by weight, and more preferably from 1 to 10% by weight, relative to the total weight of the composition.

The (c) ceramide compound may be represented by formula (I):

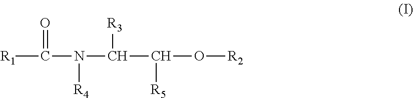

wherein

R₁ denotes:
either a saturated or unsaturated and linear or branched C₁-C₅₀, preferably C₅-C₅₀, hydrocarbon radical, it being possible for this radical to be substituted by one or more hydroxyl groups optionally esterified by an acid R₇COOH, R₇ being an optionally mono- or polyhydroxylated, saturated or unsaturated and linear or branched C₁-C₃₅ hydrocarbon radical, it being possible for the hydroxyl or hydroxyls of the R₇ radical to be esterified by an optionally mono- or polyhydroxylated, saturated or unsaturated and linear or branched C₁-C₃₅ fatty acid;
or an R″—(NR—CO)—R′ radical, in which R denotes a hydrogen atom or a mono- or polyhydroxylated, preferably monohydroxylated, C₁-C₂₀ hydrocarbon radical, R′ and R″ are hydrocarbon radicals, the sum of the carbon atoms of which is between 9 and 30, R′ being a divalent radical;
or an R₈—O—CO—(CH₂)ₚ radical, in which R₈ denotes a C₁-C₂₀ hydrocarbon radical and p is an integer varying from 1 to 12;

R₂ is chosen from a hydrogen atom, a radical of saccharide type, in particular a (glycosyl)ₙ, (galactosyl)ₘ or sulphogalactosyl radical, a sulphate or phosphate residue, a phosphorylethylamine radical and a phosphorylethylammonium radical, in which n is an integer varying from 1 to 4 and m is an integer varying from 1 to 8;

R₃ denotes a hydrogen atom or a hydroxylated or nonhydroxylated and saturated or unsaturated C₁-C₃₃ hydrocarbon radical, it being possible for the hydroxyl or hydroxyls to be esterified by an inorganic acid or an acid R₇COOH, R₇ having the same meanings as hereinabove, and it being possible for the hydroxyl or hydroxyls to be etherified by a (glycosyl), (galactosyl)ₘ, sulphogalactosyl, phosphorylethylamine or phosphorylethylammonium radical, in which n is an integer varying from 1 to 4 and m is an integer varying from 1 to 8, it also being possible for R₃ to be substituted by one or more C₁-C₁₄ alkyl radicals;

R₄ denotes a hydrogen atom, a methyl or ethyl radical, an optionally hydroxylated, saturated or unsaturated and linear or branched C₃-C₅₀ hydrocarbon radical or a —CH₂—CHOH—CH₂—O—R₆ radical, in which R₆ denotes a C₁₀-C₂₆ hydrocarbon radical, or an R₈—O—CO—(CH₂)ₚ radical, in which R₈ denotes a C₁-C₂₀ hydrocarbon radical and p is an integer varying from 1 to 12;

R₅ denotes a hydrogen atom or an optionally mono- or polyhydroxylated, saturated or unsaturated and linear or branched C₁-C₃₀ hydrocarbon radical, it being possible for the hydroxyl or hydroxyls to be etherified by a (glycosyl)ₙ, (galactosyl)ₘ, sulphogalactosyl, phosphorylethylamine or phosphorylethylammonium radical, in which n is an integer varying from 1 to 4 and m is an integer varying from 1 to 8; with the proviso that, when R₃ and R₅ denote hydrogen or when R₃ denotes hydrogen and R₅ denotes methyl, then R₄ does not denote a hydrogen atom or a methyl or ethyl radical.

It may be preferable that the (c) ceramide compound be selected from the group consisting of 2-N-linoleoylaminooctadecane-1,3-diol, 2-N-oleoylaminooctadecane-1,3-diol, 2-N-palmitoylaminooctadecane-1,3-diol, 2-N-stearoylaminooctadecane-1,3-diol, 2-N-behenoylaminooctadecane-1,3-diol, 2-N-[2-hydroxypalmitoyl]aminooctadecane-1,3-diol, 2-N-stearoylaminooctadecane-1,3,4-triol, 2-N-palmitoylaminohexadecane-1,3-diol, and mixtures thereof.

The amount of the (c) ceramide compound may range from 0.01 to 20% by weight, preferably from 0.05 to 15% by weight, and more preferably from 0.1 to 10% by weight, relative to the total weight of the composition.

The (d) compound may have a log P of from −0.7 to 6, preferably from −0.5 to 4.

It is preferable that the (d) compound be niacinamide.

The amount of the (d) compound may range from 0.01 to 20% by weight, preferably from 0.05 to 10% by weight, and more preferably from 0.1 to 5% by weight, relative to the total weight of the composition.

It may be preferable that the composition according to the present invention further comprise at least one anionic surfactant.

The present invention also relates to a cosmetic process for treating a keratin substance such as the skin, the hair, mucous membranes, the nails, the eyelashes, the eyebrows and the scalp, comprising the step of applying the composition according to the present invention to the keratin substance.

### BRIEF DESCRIPTION OF DRAWINGS

FIG. 1 shows the penetration amount of active ingredient with regard to the compositions according to Example 1 as well as Comparative Examples 1 and 2.

### BEST MODE FOR CARRYING OUT THE INVENTION

After diligent research, the inventors have found that it is possible to provide a composition in the form of a nano- or micro-emulsion with a transparent or slightly translucent, preferably transparent, aspect of the emulsion, which can further enhance or improve the penetration of a ceramide compound into a keratin substance such as the skin.

Thus, one aspect of the present invention is a composition in the form of a nano- or micro-emulsion, comprising:
(a) at least one oil;
(b) at least one nonionic surfactant with an HLB value of from 8.0 to 14.0, preferably from 9.0 to 13.5, and more preferably from 10.0 to 13.0;
(c) at least one ceramide compound;
(d) at least one compound selected from Vitamin B3 and derivatives thereof; and
(e) water.

The composition according to the present invention can further enhance or improve the penetration of a ceramide compound into a keratin substance such as the skin, as compared to a composition which does not include the above (d) compound.

The composition according to the present invention including a ceramide compound is in the form of a nano- or micro-emulsion which is transparent or slightly translucent, preferably transparent. Since the composition according to the present invention can have transparent or slightly translucent aspects, the composition according to the present invention can be preferably used for lotions and the like.

Further, as the dispersed phase is finely dispersed in the composition according to the present invention, the composition according to the present invention can also provide an excellent feeling during use, such as a moisturizing and wet feeling, as well as unique texture and increased suppleness.

Furthermore, the composition according to the present invention including a ceramide compound can be stable over time, even at an elevated temperature.

Hereafter, the composition according to the present invention and the process according to the present invention will each be described in a detailed manner.

[Composition]

(Oil)

The composition according to the present invention comprises (a) at least one oil. If two or more oils are used, they may be the same or different.

Here, “oil” means a fatty compound or substance which is in the form of a liquid or a paste (non-solid) at room temperature (25° C.) under atmospheric pressure (760 mmHg). As the oils, those generally used in cosmetics can be used alone or in combination thereof. These oils may be volatile or non-volatile.

The (a) oil may be a non-polar oil such as a hydrocarbon oil, a silicone oil, or the like; a polar oil such as a plant or animal oil and an ester oil or an ether oil; or a mixture thereof.

The (a) oil may be selected from the group consisting of oils of plant or animal origin, synthetic oils, silicone oils, hydrocarbon oils, and fatty alcohols.

As examples of plant oils, mention may be made of, for example, linseed oil, camellia oil, macadamia nut oil, corn oil, mink oil, olive oil, avocado oil, sasanqua oil, castor oil, safflower oil, jojoba oil, sunflower oil, almond oil, rapeseed oil, sesame oil, soybean oil, peanut oil, and mixtures thereof.

As examples of animal oils, mention may be made of, for example, squalene and squalane.

As examples of synthetic oils, mention may be made of alkane oils such as isododecane and isohexadecane, ester oils, ether oils, and artificial triglycerides.

The ester oils are preferably liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms of the esters being greater than or equal to 10.

Preferably, for the esters of monoalcohols, at least one from among the alcohol and the acid from which the esters of the present invention are derived is branched.

Among the monoesters of monoacids and of monoalcohols, mention may be made of ethyl palmitate, ethyl hexyl palmitate, isopropyl palmitate, dicaprylyl carbonate, alkyl myristates such as isopropyl myristate or ethyl myristate, isocetyl stearate, 2-ethylhexyl isononanoate, isononyl isononanoate, isodecyl neopentanoate, and isostearyl neopentanoate.

Esters of C₄-C₂₂ dicarboxylic or tricarboxylic acids and of C₁-C₂₂ alcohols, and esters of monocarboxylic, dicarboxylic, or tricarboxylic acids and of non-sugar C₄-C₂₆ dihydroxy, trihydroxy, tetrahydroxy, or pentahydroxy alcohols may also be used.

Mention may especially be made of: diethyl sebacate; isopropyl lauroyl sarcosinate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; diethylene glycol diisononanoate.

As ester oils, one can use sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which comprise at least 4 carbon atoms. These sugars may be monosaccharides, oligosaccharides, or polysaccharides.

Examples of suitable sugars that may be mentioned include sucrose (or saccharose), glucose, galactose, ribose, fucose, maltose, fructose, mannose, arabinose, xylose, and lactose, and derivatives thereof, especially alkyl derivatives, such as methyl derivatives, for instance methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may have one to three conjugated or non-conjugated carbon-carbon double bonds.

The esters according to this variant may also be selected from monoesters, diesters, triesters, tetraesters, and polyesters, and mixtures thereof.

These esters may be, for example, oleates, laurates, palmitates, myristates, behenates, cocoates, stearates, linoleates, linolenates, caprates, and arachidonates, or mixtures thereof such as, especially, oleopalmitate, oleostearate, and palmitostearate mixed esters, as well as pentaerythrityl tetraethyl hexanoate.

More particularly, use is made of monoesters and diesters and especially sucrose, glucose, or methylglucose monooleates or dioleates, stearates, behenates, oleopalmitates, linoleates, linolenates, and oleostearates.

An example that may be mentioned is the product sold under the name Glucate® DO by the company Amerchol, which is a methylglucose dioleate.

As examples of preferable ester oils, mention may be made of, for example, diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, dicaprylyl carbonate, isopropyl lauroyl sarcosinate, isononyl isononanoate, ethylhexyl palmitate, isohexyl laurate, hexyl laurate, isocetyl stearate, isopropyl isostearate, isopropyl myristate, isodecyl oleate, glyceryl tri(2-ethylhexanoate), pentaerythrithyl tetra(2-ethylhexanoate), 2-ethylhexyl succinate, diethyl sebacate, and mixtures thereof.

As examples of artificial triglycerides, mention may be made of, for example, capryl caprylyl glycerides, glyceryl trimyristate, glyceryl tripalmitate, glyceryl trilinolenate, glyceryl trilaurate, glyceryl tricaprate, glyceryl tricaprylate, glyceryl tri(caprate/caprylate), and glyceryl tri(caprate/caprylate/linolenate).

As examples of silicone oils, mention may be made of, for example, linear organopolysiloxanes such as dimethylpolysiloxane, methylphenylpolysiloxane, methylhydrogenpolysiloxane, and the like; cyclic organopolysiloxanes such as cyclohexasiloxane, octamethylcyclotetrasiloxane, decamethylcyclopentasiloxane, dodecamethylcyclohexasiloxane, and the like; and mixtures thereof.

Preferably, the silicone oil is chosen from liquid polydialkylsiloxanes, especially liquid polydimethylsiloxanes (PDMS) and liquid polyorganosiloxanes comprising at least one aryl group.

These silicone oils may also be organomodified. The organomodified silicones that can be used in accordance with the present invention are silicone oils as defined above and comprise in their structure one or more organofunctional groups attached via a hydrocarbon-based group.

Organopolysiloxanes are defined in greater detail in Walter Noll's Chemistry and Technology of Silicones (1968), Academic Press. They may be volatile or non-volatile.

When they are volatile, the silicones are more particularly chosen from those having a boiling point of between 60° C. and 260° C., and even more particularly from:
(i) cyclic polydialkylsiloxanes comprising from 3 to 7 and preferably 4 to 5 silicon atoms. These are, for example, octamethylcyclotetrasiloxane sold in particular under the name Volatile Silicone® 7207 by Union Carbide or Silbione® 70045 V2 by Rhodia, decamethylcyclopentasiloxane sold under the name Volatile Silicone® 7158 by Union Carbide, Silbione® 70045 V5 by Rhodia, and dodecamethylcyclopentasiloxane sold under the name Silsoft 1217 by Momentive Performance Materials, and mixtures thereof. Mention may also be made of cyclocopolymers of the type such as dimethylsiloxane/methylalkylsiloxane, such as Silicone Volatile® FZ 3109 sold by the company Union Carbide, of the formula:

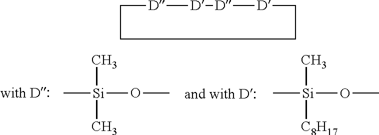

Mention may also be made of mixtures of cyclic polydialkylsiloxanes with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane; and
(ii) linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, January 76, pp. 27-32, Todd & Byers, Volatile Silicone Fluids for Cosmetics. The viscosity of the silicones is measured at 25° C. according to ASTM standard 445 Appendix C.

Non-volatile polydialkylsiloxanes may also be used. These non-volatile silicones are more particularly chosen from polydialkylsiloxanes, among which mention may be made mainly of polydimethylsiloxanes containing trimethylsilyl end groups.

Among these polydialkylsiloxanes, mention may be made, in a non-limiting manner, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a Viscosity of 60 000 mm²/s; and
the Viscasil® oils from General Electric and certain oils of the SF series (SF 96, SF 18) from General Electric.

Mention may also be made of polydimethylsiloxanes containing dimethylsilanol end groups known under the name dimethiconol (CTFA), such as the oils of the 48 series from the company Rhodia.

Among the silicones containing aryl groups, mention may be made of polydiarylsiloxanes, especially polydiphenylsiloxanes and polyalkylarylsiloxanes such as phenyl silicone oil.

The phenyl silicone oil may be chosen from the phenyl silicones of the following formula:

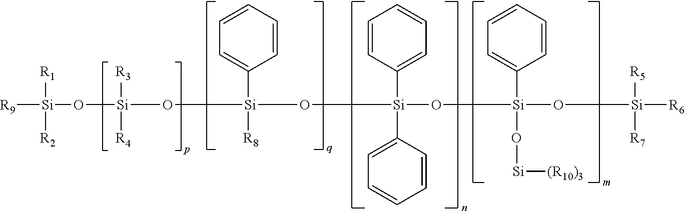

in which

R₁ to R₁₀, independently of each other, are saturated or unsaturated, linear, cyclic or branched C₁-C₃₀ hydrocarbon-based radicals, preferably C₁-C₁₂ hydrocarbon-based radicals, and more preferably C₁-C₆ hydrocarbon-based radicals, in particular methyl, ethyl, propyl, or butyl radicals, and

m, n, p, and q are, independently of each other, integers of 0 to 900 inclusive, preferably 0 to 500 inclusive, and more preferably 0 to 100 inclusive,

with the proviso that the sum n+m+q is other than 0.

Examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
certain oils of the SF series from General Electric, such as SF 1023, SF 1154, SF 1250, and SF 1265.

As the phenyl silicone oil, phenyl trimethicone (R₁ to R₁₀ are methyl; p, q, and n=0; m=1 in the above formula) is preferable.

The organomodified liquid silicones may especially contain polyethyleneoxy and/or polypropyleneoxy groups. Mention may thus be made of the silicone KF-6017 proposed by Shin-Etsu, and the oils Silwet® L722 and L77 from the company Union Carbide.

Hydrocarbon oils may be chosen from:
linear or branched, optionally cyclic, C₆-C₁₆ lower alkanes. Examples that may be mentioned include hexane, undecane, dodecane, tridecane, and isoparaffins, for instance isohexadecane, isododecane, and isodecane; and
linear or branched hydrocarbons containing more than 16 carbon atoms, such as liquid paraffins, liquid petroleum jelly, polydecenes and hydrogenated polyisobutenes such as Parleam®, and squalane.

As preferable examples of hydrocarbon oils, mention may be made of, for example, linear or branched hydrocarbons such as isohexadecane, isododecane, squalane, mineral oil (e.g., liquid paraffin), paraffin, vaseline or petrolatum, naphthalenes, and the like; hydrogenated polyisobutene, isoeicosan, and decene/butene copolymer; and mixtures thereof.

The term “fatty” in the fatty alcohol means the inclusion of a relatively large number of carbon atoms. Thus, alcohols which have 4 or more, preferably 6 or more, and more preferably 12 or more carbon atoms are encompassed within the scope of fatty alcohols. The fatty alcohol may be saturated or unsaturated. The fatty alcohol may be linear or branched.

The fatty alcohol may have the structure R—OH wherein R is chosen from saturated and unsaturated, linear and branched radicals containing from 4 to 40 carbon atoms, preferably from 6 to 30 carbon atoms, and more preferably from 12 to 20 carbon atoms. In at least one embodiment, R may be chosen from C₁₂-C₂₀ alkyl and C₁₂-C₂₀ alkenyl groups. R may or may not be substituted with at least one hydroxyl group.

As examples of the fatty alcohol, mention may be made of lauryl alcohol, cetyl alcohol, stearyl alcohol, isostearyl alcohol, behenyl alcohol, undecylenyl alcohol, myristyl alcohol, octyldodecanol, hexyldecanol, oleyl alcohol, linoleyl alcohol, palmitoleyl alcohol, arachidonyl alcohol, erucyl alcohol, and mixtures thereof.

It is preferable that the fatty alcohol be a saturated fatty alcohol.

Thus, the fatty alcohol may be selected from straight or branched, saturated or unsaturated C₆-C₃₀ alcohols, preferably straight or branched, saturated C₆-C₃₀ alcohols, and more preferably straight or branched, saturated C₁₂-C₂₀ alcohols.

The term “saturated fatty alcohol” here means an alcohol having a long aliphatic saturated carbon chain. It is preferable that the saturated fatty alcohol be selected from any linear or branched, saturated C₆-C₃₀ fatty alcohols. Among the linear or branched, saturated C₆-C₃₀ fatty alcohols, linear or branched, saturated C₁₂-C₂₀ fatty alcohols may preferably be used. Any linear or branched, saturated C₁₆-C₂₀ fatty alcohols may be more preferably used. Branched C₁₆-C₂₀ fatty alcohols may be even more preferably used.

As examples of saturated fatty alcohols, mention may be made of lauryl alcohol, cetyl alcohol, stearyl alcohol, isostearyl alcohol, behenyl alcohol, undecylenyl alcohol, myristyl alcohol, octyldodecanol, hexyldecanol, and mixtures thereof. In one embodiment, cetyl alcohol, stearyl alcohol, octyldodecanol, hexyldecanol, or a mixture thereof (e.g., cetearyl alcohol) as well as behenyl alcohol, can be used as a saturated fatty alcohol.

According to at least one embodiment, the fatty alcohol used in the composition according to the present invention is preferably chosen from cetyl alcohol, octyldodecanol, hexyldecanol, and mixtures thereof.

It is also preferable that the (a) oil be chosen from oils with molecular weight below 600 g/mol.

Preferably, the (a) oil has a low molecular weight such as below 600 g/mol, chosen among ester oils with a short hydrocarbon chain or chains (C₁-C₁₂) (e.g., isopropyl myristate, isopropyl palmitate, isononyl isononanoate, and ethyl hexyl palmitate), hydrocarbon oils (e.g., isododecane, isohexadecane, and squalane), branched and/or unsaturated fatty alcohol (C₁₂-C₃₀) type oils such as octyldodecanol and oleyl alcohol, and ether oils such as dicaprylylether.

It is preferable that the (a) oil be chosen from ester oils, hydrocarbon oils, fatty alcohols, and mixtures thereof.

The amount of the (a) oil(s) in the composition according to the present invention may range from 0.1% to 30% by weight, preferably from 0.5% to 20% by weight, more preferably from 1% to 10% by weight, even more preferably from 2% to 8% by weight, and even further more preferably from 3% to 5% by weight, relative to the total weight of the composition.

The (a) oil(s) can form a fatty phase of the composition according to the present invention.

If the composition according to the present invention is in the form of an O/W emulsion, the (a) oil in the composition according to the present invention can form dispersed fatty phases in the O/W emulsion.

The fatty phase may include other oily ingredients such as at least one organic UV filter.

The amount of the fatty phase in the composition according to the present invention may range from 0.1% to 40% by weight, preferably from 0.5% to 30% by weight, more preferably from 1% to 20% by weight, even more preferably from 2% to 10% by weight, and even further more preferably from 3 to 8% by weight, relative to the total weight of the composition.

(Nonionic Surfactant)

The composition according to the present invention comprises at least one specific nonionic surfactant. A single type of the specific nonionic surfactant may be used, but two or more different types of the specific nonionic surfactant may be used in combination.

The specific nonionic surfactant has an HLB (Hydrophilic Lipophilic Balance) value of from 8.0 to 14, preferably from 9.0 to 13.5, and more preferably from 10.0 to 13.0. If two or more nonionic surfactants are used, the HLB value is determined by the weight average of the HLB values of all the nonionic surfactants.

The (b) nonionic surfactant with an HLB value of from 8.0 to 14, preferably from 9.0 to 13.5, and more preferably from 10.0 to 13.0 may be chosen from:
(1) surfactants that are fluid at a temperature of less than or equal to 45° C., chosen from the esters of at least one polyol chosen from the group formed by polyethylene glycol comprising from 1 to 60 ethylene oxide units, sorbitan, glycerol comprising from 2 to ethylene oxide units, polyglycerols comprising from 2 to 12 glycerol units, and of at least one fatty acid comprising at least one saturated or unsaturated, linear or branched C₈-C₂₂ alkyl chain,
(2) mixed esters of fatty acid or of fatty alcohol, of carboxylic acid and of glycerol,
(3) fatty acid esters of sugars and fatty alcohol ethers of sugars,
(4) surfactants that are solid at a temperature of less than or equal to 45° C., chosen from fatty esters of glycerol, fatty esters of sorbitan and oxyethylenated fatty esters of sorbitan, ethoxylated fatty ethers and ethoxylated fatty esters,
(5) block copolymers of ethylene oxide and of propylene oxide,
(6) polyoxyethylenated (1-40 EO) and polyoxypropylenated (1-30 PO) alkyl (C₁₆-C₃₀) ethers, and
(7) silicone surfactants.

The surfactants (1) that are fluid at a temperature of less than or equal to 45° C. may be, in particular:
the isostearate of polyethylene glycol of molecular weight 400, sold under the name PEG-400 by the company Unichema;
diglyceryl isostearate, sold by the company Solvay;
glyceryl laurate comprising 2 glycerol units, sold by the company Solvay;
sorbitan oleate, sold under the name Span 80 by the company ICI;
sorbitan isostearate, sold under the name Nikkol SI 1 OR by the company Nikko; and
α-butylglucoside cocoate or α-butylglucoside caprate, sold by the company Ulice.

The (2) mixed esters of fatty acid or of fatty alcohol, of carboxylic acid and of glycerol, which can be used as the above nonionic surfactant, may be chosen in particular from the group comprising mixed esters of fatty acid or of fatty alcohol with an alkyl chain containing from 8 to 22 carbon atoms, and of α-hydroxy acid and/or of succinic acid, with glycerol. The α-hydroxy acid may be, for example, citric acid, lactic acid, glycolic acid or malic acid, and mixtures thereof.

The alkyl chain of the fatty acids or alcohols from which are derived the mixed esters which can be used in the nanoemulsion of the invention may be linear or branched, and saturated or unsaturated. They may especially be stearate, isostearate, linoleate, oleate, behenate, arachidonate, palmitate, myristate, laurate, caprate, isostearyl, stearyl, linoleyl, oleyl, behenyl, myristyl, lauryl or capryl chains, and mixtures thereof.

As examples of mixed esters which can be used in the nanoemulsion of the invention, mention may be made of the mixed ester of glycerol and of the mixture of citric acid, lactic acid, linoleic acid and oleic acid (CTFA name: Glyceryl citrate/lactate/linoleate/oleate) sold by the company Hills under the name Imwitor 375; the mixed ester of succinic acid and of isostearyl alcohol with glycerol (CTFA name: Isostearyl diglyceryl succinate) sold by the company Hills under the name Imwitor 780 K; the mixed ester of citric acid and of stearic acid with glycerol (CTFA name: Glyceryl stearate citrate) sold by the company Hills under the name Imwitor 370; the mixed ester of lactic acid and of stearic acid with glycerol (CTFA name: Glyceryl stearate lactate) sold by the company Danisco under the name Lactodan B30 or Rylo LA30.

The (3) fatty acid esters of sugars, which can be used as the above nonionic surfactant, may preferably be solid at a temperature of less than or equal to 45° C. and may be chosen in particular from the group comprising esters or mixtures of esters of C₈-C₂₂ fatty acid and of sucrose, of maltose, of glucose or of fructose, and esters or mixtures of esters of C₁₄-C₂₂ fatty acid and of methylglucose.

The C₈-C₂₂ or C₁₄-C₂₂ fatty acids forming the fatty unit of the esters which can be used in the present invention comprise a saturated or unsaturated linear alkyl chain containing, respectively, from 8 to 22 or from 14 to 22 carbon atoms. The fatty unit of the esters may be chosen in particular from stearates, behenates, arachidonates, palmitates, myristates, laurates and caprates, and mixtures thereof. Stearates are preferably used.

As examples of esters or mixtures of esters of fatty acid and of sucrose, of maltose, of glucose or of fructose, mention may be made of sucrose monostearate, sucrose distearate and sucrose tristearate and mixtures thereof, such as the products sold by the company Croda under the name Crodesta F50, F70, F110 and F160; and examples of esters or mixtures of esters of fatty acid and of methylglucose which may be mentioned are methylglucose polyglyceryl-3 distearate, sold by the company Goldschmidt under the name Tego-care 450. Mention may also be made of glucose or maltose monoesters such as methyl o-hexadecanoyl-6-D-glucoside and o-hexadecanoyl-6-D-maltoside.

The (3) fatty alcohol ethers of sugars, which can be used as the above nonionic surfactant, may be solid at a temperature of less than or equal to 45° C. and may be chosen in particular from the group comprising ethers or mixtures of ethers of C₈-C₂₂ fatty alcohol and of glucose, of maltose, of sucrose or of fructose, and ethers or mixtures of ethers of a C₁₄-C₂₂ fatty alcohol and of methylglucose. These are in particular alkylpolyglucosides.

The C₈-C₂₂ or C₁₄-C₂₂ fatty alcohols forming the fatty unit of the ethers which may be used in the nanoemulsion of the invention comprise a saturated or unsaturated, linear alkyl chain containing, respectively, from 8 to 22 or from 14 to 22 carbon atoms. The fatty unit of the ethers may be chosen in particular from decyl, cetyl, behenyl, arachidyl, stearyl, palmityl, myristyl, lauryl, capryl and hexadecanoyl units, and mixtures thereof, such as cetearyl.

As examples of fatty alcohol ethers of sugars, mention may be made of alkylpolyglucosides such as decylglucoside and laurylglucoside, which is sold, for example, by the company Henkel under the respective names Plantaren 2000 and Plantaren 1200, cetostearyl glucoside optionally as a mixture with cetostearyl alcohol, sold for example, under the name Montanov 68 by the company SEPPIC, under the name Tego-care CG90 by the company Goldschmidt and under the name Emulgade KE3302 by the company Henkel, as well as arachidyl glucoside, for example in the form of a mixture of arachidyl alcohol and behenyl alcohol and arachidyl glucoside, sold under the name Montanov 202 by the company SEPPIC.

The surfactant used more particularly is sucrose monostearate, sucrose distearate or sucrose tristearate and mixtures thereof, methylglucose polyglyceryl-3 distearate and alkylpolyglucosides.

The (4) fatty esters of glycerol which may be used as the above nonionic surfactant, which are solid at a temperature of less than or equal to 45° C., may be chosen in particular from the group comprising esters formed from at least one acid comprising a saturated linear alkyl chain containing from 12 to 22 carbon atoms and from 1 to 12 glycerol units. One or more of these fatty esters of glycerol may be used in the present invention.

These esters may be chosen in particular from stearates, behenates, arachidates and palmitates, and mixtures thereof. Stearates and palmitates are preferably used.

As examples of surfactants which can be used in the present invention, mention may be made of decaglyceryl monostearate, distearate, tristearate and pentastearate (CTFA names: Polyglyceryl-10 stearate, Polyglyceryl-10 distearate, Polyglyceryl-10 tristearate, Polyglyceryl-10 pentastearate), such as the products sold under the respective names Nikkol Decaglyn 1-S, 2-S, 3-S and 5-S by the company Nikko, and diglyceryl monostearate (CTFA name: Polyglyceryl-2 stearate), such as the product sold by the company Nikko under the name Nikkol DGMS.

The (4) fatty esters of sorbitan which may be used as the above nonionic surfactant, which are solid at a temperature of less than or equal to 45° C., may be chosen from the group comprising C₁₆-C₂₂ fatty acid esters of sorbitan and oxyethylenated C₁₆-C₂₂ fatty acid esters of sorbitan. 50 They are formed from at least one fatty acid comprising at least one saturated linear alkyl chain containing, respectively, from 16 to 22 carbon atoms, and from sorbitol or from ethoxylated sorbitol. The oxyethylenated esters generally comprise from 1 to 100 ethylene glycol units and preferably from 2 to 40 ethylene oxide (EO) units.

These esters may be chosen in particular from stearates, behenates, arachidates, palmitates, and mixtures thereof. Stearates and palmitates are preferably used.

As examples of the above nonionic surfactant which can be used in the present invention, mention may be made of sorbitan monostearate (CTFA name: sorbitan stearate), sold by the company ICI under the name Span 60, sorbitan monopalmitate (CTFA name: sorbitan palmitate), sold by the company ICI under the name Span 40, and sorbitan tristearate 20 EO (CTFA name: polysorbate 65), sold by the company ICI under the name Tween 65.

The (4) ethoxylated fatty ethers that are solid at a temperature of less than or equal to 45° C., which may be used as the above nonionic surfactant, are preferably ethers formed from 1 to 100 ethylene oxide units and from at least one fatty alcohol chain containing from 16 to 22 carbon atoms. The fatty chain of the ethers may be chosen in particular from behenyl, arachidyl, stearyl and cetyl units, and mixtures thereof, such as cetearyl. Examples of ethoxylated fatty ethers which may be mentioned are behenyl alcohol ethers comprising 5, 10, and 30 ethylene oxide units (CTFA names: beheneth-5, beheneth-10, beheneth-20, beheneth-30), such as the products sold under the names Nikkol BB5, BB10, BB20 and BB30 by the company Nikko, and stearyl alcohol ether comprising 2 ethylene oxide units (CTFA name: steareth-2), such as the product sold under the name Brij 72 by the company ICI.

The (4) ethoxylated fatty esters that are solid at a temperature of less than or equal to 45° C., which may be used as the above nonionic surfactant, are esters formed from 1 to 100 ethylene oxide units and from at least one fatty acid chain containing from 16 to 22 carbon atoms. The fatty chain in the esters may be chosen in particular from stearate, behenate, arachidate and palmitate units, and mixtures thereof. Examples of ethoxylated fatty esters which may be mentioned are the ester of stearic acid comprising 40 ethylene oxide units, such as the product sold under the name Myrj 52 (CTFA name: PEG-40 stearate) by the company ICI, as well as the ester of behenic acid comprising 8 ethylene oxide units (CTFA name: PEG-8 behenate), such as the product sold under the name Compritol HD5 ATO by the company Gattefosse.

The (5) block copolymers of ethylene oxide and of propylene oxide, which may be used as surfactants in the nanoemulsion according to the invention, may be chosen in particular from block copolymers of formula (I):

HO(C₂H₄₀)ₓ(C₃H₆₀)_{y}(C₂H₄₀)_{z}H (I)

in which x, y and z are integers such that x+z ranges from 2 to 100 and y ranges from 14 to 60, and mixtures thereof, and more particularly from the block copolymers of formula (I) having an HLB value ranging from 8.0 to 14.

The (6) polyoxyethylenated (1-40 EO) and polyoxypropylenated (1-30 PO) alkyl (C₁₆-C₃₀) ethers which may be used as surfactants in the nanoemulsion according to the invention, may be selected from the group consisting of:

PPG-6 Decyltetradeceth-30; Polyoxyethlene (30) Polyoxypropylene (6) Tetradecyl Ether such 50 as those sold as Nikkol PEN-4630 from Nikko Chemicals Co.,

PPG-6 Decyltetradeceth-12; Polyoxyethylene (12) Polyoxypropylene (6) Tetradecyl Ether such as those sold as Nikkol PEN-4612 from Nikko Chemicals Co.,

PPG-13 Decyltetradeceth-24; Polyoxyethylene (24) Polyoxypropylene (13) Decyltetradecyl Ether such as those sold as UNILUBE 50MT-2200B from NOF Corporation, PPG-6 Decyltetradeceth-20; Polyoxyethylene (20) Polyoxypropylene (6) Decyltetradecyl Ether such as those sold as Nikkol PEN-4620 from Nikko Chemicals Co.,

PPG-4 Ceteth-1; Polyoxyethylene (1) Polyoxypropylene (4) Cetyl Ether such as those sold as Nikkol PBC-31 from Nikko Chemicals Co.,

PPG-8 Ceteth-1; Polyoxyethylene (1) Polyoxypropylene (8) Cetyl Ether such as those sold as Nikkol PBC-41 from Nikko Chemicals Co.,

PPG-4 Ceteth-10; Polyoxyethylene (10) Polyoxypropylene (4) Cetyl Ether such as those sold as Nikkol PBC-33 from Nikko Chemicals Co.,

PPG-4 Ceteth-20; Polyoxyethylene (20) Polyoxypropylene (4) Cetyl Ether such as those sold as Nikkol PBC-34 from Nikko Chemicals Co.,

PPG-5 Ceteth-20; Polyoxyethylene (20) Polyoxypropylene (5) Cetyl Ether such as those sold as Procetyl AWS from Croda Inc.,

PPG-8 Ceteth-20; Polyoxyethylene (20) Polyoxypropylene (8) Cetyl Ether such as those sold as Nikkol PBC-44 from Nikko Chemicals Co., and

PPG-23 Steareth-34; Polyoxyethylene Polyoxypropylene Stearyl Ether (34 EO) (23 PO) such as those sold as Unisafe 34S-23 from Pola Chemical Industries. They can provide a composition with long-term stability, even though the temperature of the composition is increased and decreased in a relatively short period of time.

It is more preferable that the polyoxyethylenated (1-40 EO) and polyoxypropylenated (1-30 PO) alkyl (C₁₆-C₃₀) ethers be (15-40 EO) and polyoxypropylenated (5-30 PO) alkyl (C₁₆-C₂₄) ethers, which could be selected from the group consisting of PPG-6 Decyltetradeceth-30, PPG-13 Decyltetradeceth-24, PPG-6 Decyltetradeceth-20, PPG-5 Ceteth-20, PPG-8 Ceteth-20, and PPG-23 Steareth-34.

It is most preferable that the polyoxyethylenated (1-40 EO) and polyoxypropylenated (1-30 PO) alkyl (C₁₆-C₃₀) ethers be (15-40 EO) and polyoxypropylenated (5-30 PO) alkyl (C₁₆-C₂₄) ethers, which could be selected from the group consisting of PPG-6 Decyltetradeceth-30, PPG-13 Decyltetradeceth-24, PPG-5 Ceteth-20, and PPG-8 Ceteth-20. They can also provide a composition with long-term transparency.

As (7) silicone surfactants which can be used according to the present invention, mention may be made of those disclosed in documents U.S. Pat. Nos. 5,364,633 and 5,411,744.

The (7) silicone surfactant as the above nonionic surfactant may preferably be a compound of formula (I):

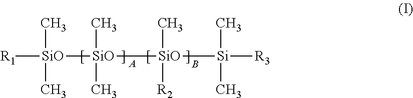

in which:

R₁, R₂ and R₃, independently of each other, represent a C₁-C₆ alkyl radical or a radical —(CH₂)ₓ—(OCH₂CH₂)_{y}—(OCH₂CH₂CH₂)_{z}—OR₄, at least one radical R₁, R₂ or R₃ not being an alkyl radical; R₄ being a hydrogen, an alkyl radical or an acyl radical;

A is an integer ranging from 0 to 200;

B is an integer ranging from 0 to 50; with the proviso that A and B are not simultaneously equal to zero;

x is an integer ranging from 1 to 6;

y is an integer ranging from 1 to 30;

z is an integer ranging from 0 to 5.

According to one preferred embodiment of the invention, in the compound of formula (I), the alkyl radical is a methyl radical, x is an integer ranging from 2 to 6 and y is an integer ranging from 4 to 30.

As examples of silicone surfactants of formula (I), mention may be made of the compounds of formula (II):

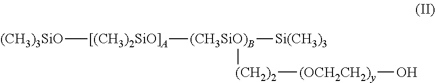

in which A is an integer ranging from 20 to 105, B is an integer ranging from 2 to 10 and y is an integer ranging from 10 to 20.

As examples of silicone surfactants of formula (I), mention may also be made of the compounds of formula (III):

H—(OCH₂CH₂)_{y}—(CH₂)₃—[(CH₃)₂SiO]_{A′}—(CH₂)₃—(OCH₂CH₂)_{y}—OH (III)

in which A′ and y are integers ranging from 10 to 20.

Compounds of the invention which may be used are those sold by the company Dow Corning under the names DC 5329, DC 7439-146, DC 2-5695 and Q4-3667. The compounds DC 5329, DC 7439-146 and DC 2-5695 are compounds of formula (II) in which, respectively, A is 22, B is 2 and y is 12; A is 103, B is 10 and y is 12; and A is 27, B is 3 and y is 12.

The compound Q4-3667 is a compound of formula (III) in which A is 15 and y is 13.

It is preferable that the (b) nonionic surfactant with an HLB value of from 8.0 to 14, preferably from 9.0 to 13.5, and more preferably from 10.0 to 13.0 be chosen from:
polyethylene glycol isostearate or oleate (8 to 10 mol of ethylene oxide),
polyethylene glycol isocetyl, behenyl ether or isostearyl ether (8 to 10 mol of ethylene oxide),
polyglyceryl monolaurate or dilaurate comprising 3 to 6 glycerol units,
polyglyceryl mono(iso)stearate comprising 3 to 6 glycerol units,
polyglyceryl monooleate comprising 3 to 6 glycerol units, and
polyglyceryl dioleate comprising 3 to 6 glycerol units.

According to a preferable embodiment of the present invention, the (b) nonionic surfactant with an HLB value of from 8.0 to 14, preferably from 9.0 to 13.5, and more preferably from 10.0 to 13.0, is selected from polyglyceryl fatty acid esters and mono- or poly-oxyalkylenated fatty acid esters.

It is preferable that the polyglyceryl fatty acid ester comprise esters of a fatty acid and polyglycerine containing 70% or more of polyglycerine whose polymerization degree is 4 or more, preferably esters of a fatty acid and polyglycerine containing equal to or more than 60% of polyglycerine whose polymerization degree is between 4 and 11, and more preferably esters of a fatty acid and polyglycerine containing equal to or more than 30% of polyglycerine whose polymerization degree is 5.

The polyglyceryl fatty acid ester may be chosen from the mono, di and tri esters of saturated or unsaturated acid, preferably saturated acid, including 2 to 30 carbon atoms, preferably 6 to carbon atoms, and more preferably 8 to 30 carbon atoms, such as lauric acid, oleic acid, stearic acid, isostearic acid, capric acid, caprylic acid, and myristic acid.

It is preferable that the polyglyceryl fatty acid ester be selected from the group consisting of polyglyceryl (PG)-4 laurate, PG-5 laurate, PG5 dilaurate, PG-5 oleate, PG-5 dioleate, PG-6 tricaprylate, PG-5 myristate, PG-5 trimyristate, PG-5 stearate, PG-5 isostearate, PG-5 trioleate, PG-6 caprylate, and PG-6 tricaprylate.

It is preferable that the mono- or poly-oxyalkylenated fatty acid ester have a (poly)oxyalkylene moiety derived from 1 to 20 oxyalkylenes, preferably from 3 to 15 oxyalkylenes, and more preferably 8 to 10 oxyalkylenes.

The oxyalkylene moiety may be derived from alkylene glycols such as ethyleneglycol, propylene glycol, butyleneglycol, pentyleneglycol, hexyleneglycol, and the like. The oxyalkylene moiety may contain a number of moles of ethylene oxide and/or of propylene oxide of between 1 and 100 and preferably between 2 and 50. Advantageously, the nonionic surfactants do not comprise any oxypropylene units.

The mono- or poly-oxyalkylenated fatty acid ester may be chosen from the mono and di esters of saturated or unsaturated acid, preferably saturated acid, including 2 to 30 carbon atoms, preferably 6 to 30 carbon atoms, and more preferably 8 to 30 carbon atoms, such as lauric acid, oleic acid, stearic acid, isostearic acid, capric acid, caprylic acid, and myristic acid.

Examples of mono- or poly-oxyalkylenated fatty acid esters that may be mentioned include esters of saturated or unsaturated, linear or branched, C₂-C₃₀, preferably C₆-C₃₀ and more preferably C₈-C₂₂ acids and of polyethylene glycols.

Examples of mono- or poly-oxyalkylenated fatty acid esters that may be mentioned include the adducts of ethylene oxide with esters of lauric acid, myristic acid, palmitic acid, stearic acid, isostearic acid, oleic acid or behenic acid, and mixtures thereof, especially those containing from 8 to 20 oxyethylene groups, such as PEG-8 to PEG-20 laurate (as the CTFA names: PEG-8 laurate to PEG-20 laurate); PEG-8 to PEG-20 myristate (as the CTFA names: PEG-8 mysistate to PEG-20 mysistate); PEG-8 to PEG-20 palmitate (as the CTFA names: PEG-8 palmitate to PEG-20 palmitate); PEG-8 to PEG-20 stearate (as the CTFA names: PEG-8 stearate to PEG-20 stearate); PEG-8 to PEG-20 isostearate (as the CTFA names: PEG-8 isostearate to PEG-20 isostearate); PEG-8 to PEG-20 oleate (as the CTFA names: PEG-8 oleate to PEG-20 oleate); PEG-8 to PEG-20 behenate (as the CTFA names: PEG-8 behenate to PEG-20 behenate); and mixtures thereof.

It is preferable that the polyglycol fatty acid ester be selected from the group consisting of 50 PEG-8 isostearate, PEG-8 stearate, PEG-10 isostearate, PEG-10 oleate, PEG-10 isocetyl ether, PEG-10 behenyl ether or PEG-10 isostearyl ether and a mixture thereof.

Preferred nonionic surfactants are polyglyceryl fatty acid esters.

The amount of the (b) nonionic surfactant(s) with an HLB value of from 8.0 to 14, preferably from 9.0 to 13.5, and more preferably from 10.0 to 13.0 may be 30% by weight or less, preferably 20% by weight or less, and more preferably 10% by weight or less, relative to the total weight of the composition according to the present invention, with the proviso that the amount of the (b) nonionic surfactant is not zero. The amount of the (b) nonionic surfactant(s) may be 0.1% by weight or more, preferably 0.5% by weight or more, and more preferably from 1% by weight or more, relative to the total weight of the composition.

Thus, the amount in the composition according to the present invention of the (b) nonionic surfactant(s) with an HLB value of from 8.0 to 14, preferably from 9.0 to 13.5, and more preferably from 10.0 to 13.0 may range from 0.1 to 30% by weight, preferably from 0.5 to 20% by weight, and more preferably from 1 to 10% by weight, relative to the total weight of the composition.

The weight ratio of the amount of the (b) nonionic surfactant(s)/the amount of the (a) oil(s) may be 2 or less, preferably from 1.5 or less, and more preferably from 1.0 or less.

(Ceramide Compound)

The composition according to the present invention comprises (c) at least one ceramide compound. A single type of ceramide compound may be used, but two or more different types of ceramide compound may be used in combination.

According to the present invention, the term “ceramide compound” is understood to mean natural or synthetic ceramides and/or glycoceramides and/or pseudoceramides and/or neoceramides.

Ceramide compounds are disclosed, for example, in Patent Applications DE 4424530, DE 4424533, DE 4402929, DE 4420736, WO 95/23807, WO 94/07844, EP-A-0 646 572, WO 95/16665, FR-2 673 179, EP-A-0 227 994, WO 94/07844, WO 94/24097 and WO 94/10131, the teachings of which are included here by way of reference.

Ceramide compounds which can be used according to the present invention include, and in fact preferably correspond to, the general formula (I):

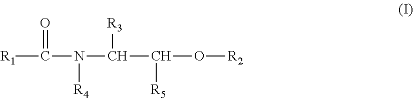

wherein

R₁ denotes:
either a saturated or unsaturated and linear or branched C₁-C₅₀, preferably C₅-C₅₀, hydrocarbon radical, it being possible for this radical to be substituted by one or more hydroxyl groups optionally esterified by an acid R₇COOH, R₇ being an optionally mono- or polyhydroxylated, saturated or unsaturated and linear or branched C₁-C₃₅ hydrocarbon radical, it being possible for the hydroxyl or hydroxyls of the R₇ radical to be esterified by an optionally mono- or polyhydroxylated, saturated or unsaturated and linear or branched C₁-C₃₅ fatty acid;
or an R″—(NR—CO)—R′ radical, in which R denotes a hydrogen atom or a mono- or polyhydroxylated, preferably monohydroxylated, C₁-C₂₀ hydrocarbon radical, R′ and R″ are hydrocarbon radicals, the sum of the carbon atoms of which is between 9 and 30, R′ being a divalent radical;
or an R₈—O—CO—(CH₂)ₚ radical, in which R₈ denotes a C₁-C₂₀ hydrocarbon radical and p is an integer varying from 1 to 12;

R₂ is chosen from a hydrogen atom, a radical of saccharide type, in particular a (glycosyl)ₙ, (galactosyl)ₘ or sulphogalactosyl radical, a sulphate or phosphate residue, a phosphorylethylamine radical and a phosphorylethylammonium radical, in which n is an integer varying from 1 to 4 and m is an integer varying from 1 to 8;

R₃ denotes a hydrogen atom or a hydroxylated or nonhydroxylated and saturated or unsaturated C₁-C₃₃ hydrocarbon radical, it being possible for the hydroxyl or hydroxyls to be esterified by an inorganic acid or an acid R₇COOH, R₇ having the same meanings as hereinabove, and it being possible for the hydroxyl or hydroxyls to be etherified by a (glycosyl)ₙ, (galactosyl)ₘ, sulphogalactosyl, phosphorylethylamine or phosphorylethylammonium radical, in which n is an integer varying from 1 to 4 and m is an integer varying from 1 to 8, it also being possible for R₃ to be substituted by one or more C₁-C₁₄ alkyl radicals;

R₄ denotes a hydrogen atom, a methyl or ethyl radical, an optionally hydroxylated, saturated or unsaturated and linear or branched C₃-C₅₀ hydrocarbon radical or a —CH₂—CHOH—CH₂—O—R₆ radical, in which R₆ denotes a C₁₀-C₂₆ hydrocarbon radical, or an R₈—O—CO—(CH₂)ₚ radical, in which R₈ denotes a C₁-C₂₀ hydrocarbon radical and p is an integer varying from 1 to 12;

R₅ denotes a hydrogen atom or an optionally mono- or polyhydroxylated, saturated or unsaturated and linear or branched C₁-C₃₀ hydrocarbon radical, it being possible for the hydroxyl or hydroxyls to be etherified by a (glycosyl)ₙ, (galactosyl)ₘ, sulphogalactosyl, phosphorylethylamine or phosphorylethylammonium radical, in which n is an integer varying from 1 to 4 and m is an integer varying from 1 to 8; with the proviso that, when R₃ and R₅ denote hydrogen or when R₃ denotes hydrogen and R₅ denotes methyl, then R₄ does not denote a hydrogen atom or a methyl or ethyl radical.

Preference is given, among the compounds of formula (I), to the ceramides and/or glycoceramides with the structure described by Downing in Journal of Lipid Research, Vol. 35, 2060-2068, 1994, or those disclosed in French Patent Application FR-2 673 179, the teachings of which are incorporated herein by reference.

It is preferable that, in the above formula (I), R₃ denote a C₁₅-C₂₆ α-hydroxyalkyl radical, the hydroxyl group optionally being esterified by a C₁₆-C₃₀ α-hydroxy acid.

The ceramide compounds which are more particularly preferred according to the invention are the compounds of formula (I) for which R₁ denotes an optionally hydroxylated and saturated or unsaturated alkyl derived from C₁₄-C₂₂ fatty acids; R₂ denotes a hydrogen atom; and R₃ denotes an optionally hydroxylated and linear C₁₁-C₁₇ radical and preferably C₁₃-C₁₅ radical. R₃ preferably denotes an α-hydroxycetyl radical and R₂, R₄ and R₅ denote a hydrogen atom.

It is preferable that the (c) ceramide compound be selected from the group consisting of 2-N-linoleoylaminooctadecane-1,3-diol, 2-N-oleoylaminooctadecane-1,3-diol, 2-N-palmitoylaminooctadecane-1,3-diol, 2-N-stearoylaminooctadecane-1,3-diol, 2-N-behenoylaminooctadecane-1,3-diol, 2-N-[2-hydroxypalmitoyl]aminooctadecane-1,3-diol, 2-N-stearoylaminooctadecane-1,3,4-triol, 2-N-palmitoylaminohexadecane-1,3-diol, and mixtures thereof.

It is also preferable that the (c) ceramide compound be chosen from bis(N-hydroxyethyl-N-cetyl)malonamide, the N-(2-hydroxyethyl)-N-(3-cetyloxy-2-hydroxypropyl)amide of cetylic acid and N-docosanoyl-N-methyl-D-glucamine.

Use may also be made of mixtures of ceramide compounds, such as, for example, the mixtures of ceramide(s) 2 and ceramide(s) 5 according to the Downing classification.

Particular use may also be made of the compounds of formula (I) for which R₁ denotes a saturated or unsaturated alkyl radical derived from C₁₂-C₂₂ fatty acids; R₂ denotes a galactosyl or sulphogalactosyl radical; and R₃ denotes a saturated or unsaturated C₁₂-C₂₂ hydrocarbon radical and preferably a —CH═CH—(CH₂)₁₂—CH₃ group.

Mention may be made, by way of example, of the product composed of a mixture of glycoceramides sold under the trade name Glycocer by Waitaki International Biosciences.

Use may also be made of the compounds of formula (I) disclosed in Patent Applications EP-A-0 227 994, EP-A-0 647 617, EP-A-0 736 522 and WO 94/07844.

Such compounds include, for example, Questamide H (bis(N-hydroxyethyl-N-cetyl)malonamide), sold by Quest, or the N-(2-hydroxyethyl)-N-(3-cetyloxy-2-hydroxy-propyl)amide of cetylic acid.

Use may also be made of N-docosanoyl-N-methyl-D-glucamine, disclosed in Patent Application WO 94/24097.

It is of course possible to use mixtures of the various ceramide compounds in the invention compositions.

The amount of the (c) ceramide compound(s) may be 20% by weight or less, preferably 15% by weight or less, and more preferably 10% by weight or less, relative to the total weight of the composition according to the present invention, with the proviso that the amount of the (c) ceramide compound is not zero. The amount of the (c) ceramide compound(s) may be 0.01% by weight or more, preferably 0.05% by weight or more, and more preferably from 0.1% by weight or more, relative to the total weight of the composition.

Thus, the amount of the (c) ceramide compound(s) may range from 0.01 to 20% by weight, preferably from 0.05 to 15% by weight, more preferably from 0.1 to 10% by weight, and further more preferably from 0.1 to 5% by weight, relative to the total weight of the composition.

The weight ratio of the amount of the (b) nonionic surfactant(s)/the amount of the (c) 50 ceramide compound(s) may be 5 or more, preferably 10 or more, more preferably 15 or more, and even more preferably 20 or more.

(Vitamin B3 and Derivatives Thereof)

The composition according to the present invention comprises (d) at least one compound selected from Vitamin B3 and derivatives thereof. A single type of the (d) compound may be used, but two or more different types of the (d) compound may be used in combination.

Vitamin B3, also called Vitamin PP, is a compound of the following formula:

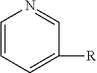

in which R may be —CONH₂ (niacinamide), —COOH (nicotinic acid or niacin), or CH₂OH (nicotinyl alcohol), —CO—NH—CH₂—COOH (nicotinuric acid) or —CO—NH—OH (niconityl hydroxamic acid). Niacinamide is preferable.

Vitamin B3 derivatives that may be mentioned include, for example, nicotinic acid esters such as tocopherol nicotinate, amides derived from niacinamide by substitution of the hydrogen groups of —CONH₂, products from reaction with carboxylic acids and amino acids, esters of nicotinyl alcohol and of carboxylic acids such as acetic acid, salicyclic acid, glycolic acid or palmitic acid.

Mention may also be made of the following derivatives: 2-chloronicotinamide, 6-methylnicotinamide, 6-aminonicotinamide, N-methylnicotinamide, N,N-dimethylnicotinamide, N-(hydroxymethyl)nicotinamide, quinolinic acid imide, nicotinanilide, N-benzylnicotinamide, N-ethylnicotinamide, nifenazone, nicotinaldehyde, isonicotinic acid, methylisonicotinic acid, thionicotinamide, nialamide, 2-mercaptonicotinic acid, nicomol and niaprazine, methyl nicotinate and sodium nicotinate.

Other Vitamin B3 derivatives that may also be mentioned include its inorganic salts, such as chlorides, bromides, iodides or carbonates, and its organic salts, such as the salts obtained by reaction with carboxylic acids, such as acetate, salicylate, glycolate, lactate, malate, citrate, mandelate, tartrate, etc.

It is preferable that the (d) compound selected from Vitamin B3 and derivatives thereof have a log P of from −0.7 to 6, and preferably from −0.5 to 4.

A log P value is a value for the base-ten logarithm of the apparent octan-1-ol/water partition coefficient. The log P values are known and are determined by a standard test which determines the concentration of the (d) compound in octan-1-ol and water. The log P may be calculated according to the method described in the article by Meylan and Howard: Atom/Fragment contribution method for estimating octanol-water partition coefficients, J. Pharm. Sci., 84: 83-92, 1995. This value may also be calculated using numerous commercially available software packages, which determine the log P as a function of the structure of a molecule. By way of example, mention may be made of the Epiwin software from the United States Environmental Agency.

The values may especially be calculated using the ACD (Advanced Chemistry Development) Solaris software V4.67; they may also be obtained from Exploring QSAR: hydrophobic, electronic and steric constants (ACS professional reference book, 1995). There is also an Internet site which provides estimated values (address: http://esc.syrres.com/interkow/kowdemo.htm).

The amount of the (d) compound(s) may be 20% by weight or less, preferably 15% by weight or less, and more preferably 10% by weight or less, relative to the total weight of the composition according to the present invention, with the proviso that the amount of the (d) compound is not zero. The amount of the (d) compound(s) may be 0.01% by weight or more, preferably 0.05% by weight or more, and more preferably from 0.1% by weight or more, relative to the total weight of the composition.

Thus, the amount in the composition according to the present invention of the (d) compound(s) selected from Vitamin B3 and derivatives thereof may range from 0.01 to 20% by weight, preferably from 0.05 to 15% by weight, more preferably from 0.1 to 10% by weight, further more preferably 0.5 to 5% by weight, relative to the total weight of the composition.

(Water)

The composition according to the present invention comprises (e) water.

The amount of water is not limited, and may be from 40 to 90% by weight, preferably from 50 to 85% by weight, and more preferably from 60 to 80% by weight, relative to the total weight of the composition.

The (e) water can form an aqueous phase of the composition according to the present invention.

If the composition according to the present invention is in the form of an O/W emulsion, the (e) water in the composition according to the present invention can form continuous aqueous phases in the O/W emulsion.

The aqueous phase may include any hydrophilic ingredients such as monohydric alcohol such as ethanol, and polyol such as glycerine, which are explained later.

The amount of the aqueous phase in the composition according to the present invention may range from 30% to 99% by weight, preferably from 40% to 98% by weight, more preferably from 50% to 95% by weight, even more preferably from 55% to 93% by weight, and even further more preferably from 60 to 90% by weight, relative to the total weight of the composition.

(Anionic Surfactant)

The composition according to the present invention may comprise (f) at least one anionic surfactant. A single type of anionic surfactant may be used, but two or more different types of anionic surfactant may be used in combination.

The (f) anionic surfactant is not limited. The (f) anionic surfactant may be chosen in particular from anionic derivatives of proteins of vegetable origin or of silk proteins, phosphates and alkyl phosphates, carboxylates, sulphosuccinates, amino acid derivatives, alkyl sulphates, alkyl ether sulphates, sulphonates, isethionates, taurates, alkyl sulphoacetates, polypeptides, anionic derivatives of alkyl polyglucosides, and their mixtures.

1) Anionic derivatives of proteins of vegetable origin are protein hydrolysates comprising a hydrophobic group, it being possible for the said hydrophobic group to be naturally present in the protein or to be added by reaction of the protein and/or of the protein hydrolysate with a hydrophobic compound. The proteins are of vegetable origin or derived from silk, and the hydrophobic group can in particular be a fatty chain, for example an alkyl chain comprising from 10 to 22 carbon atoms. Mention may more particularly be made, as anionic derivatives of proteins of vegetable origin, of apple, wheat, soybean or oat protein hydrolysates comprising an alkyl chain having from 10 to 22 carbon atoms, and their salts. The alkyl chain can in particular be a lauryl chain and the salt can be a sodium, potassium and/or ammonium salt.

Thus, mention may be made, as protein hydrolysates comprising a hydrophobic group, for example, of salts of protein hydrolysates where the protein is a silk protein modified by lauric acid, such as the product sold under the name Kawa Silk by Kawaken; salts of protein hydrolysates where the protein is a wheat protein modified by lauric acid, such as the potassium salt sold under the name Aminofoam W OR by Croda (CTFA name: potassium lauroyl wheat amino acids) and the sodium salt sold under the name Proteol LW 30 by Seppic (CTFA name: sodium lauroyl wheat amino acids); salts of protein hydrolysates where the protein is an oat protein comprising an alkyl chain having from 10 to 22 carbon atoms and more especially salts of protein hydrolysates where the protein is an oat protein modified by lauric acid, such as the sodium salt sold under the name Proteol OAT (30% aqueous solution) by Seppic (CTFA name: sodium lauroyl oat amino acids); or salts of apple protein hydrolysates comprising an alkyl chain having from 10 to 22 carbon atoms, such as the sodium salt sold under the name Proteol APL (30% aqueous/glycol solution) by Seppic (CTFA name: sodium cocoyl apple amino acids). Mention may also be made of the mixture of lauroyl amino acids (aspartic acid, glutamic acid, glycine, alanine) neutralized with sodium N-methylglycinate sold under the name Proteol SAV 50 S by Seppic (CTFA name: sodium cocoyl amino acids).

2) Mention may be made, as phosphates and alkyl phosphates, for example, of monoalkyl phosphates and dialkyl phosphates, such as lauryl monophosphate, sold under the name MAP 20® by Kao Chemicals, the potassium salt of dodecyl phosphate, the mixture of mono- and diesters (predominantly diester) sold under the name Crafol AP-31 ® by Cognis, the mixture of octyl phosphate monoester and diester, sold under the name Crafol AP-20® by Cognis, the mixture of ethoxylated (7 mol of EO) 2-butyloctyl phosphate monoester and diester, sold under the name Isofol 12 7 EO-Phosphate Ester® by Condea, the potassium or triethanolamine salt of mono(C₁₂-C₁₃)alkyl phosphate, sold under the references Arlatone MAP 230K-40® and Arlatone MAP 230T-60® by Uniqema, potassium lauryl phosphate, sold under the name Dermalcare MAP XC-99/09® by Rhodia Chimie, and potassium cetyl phosphate, sold under the name Arlatone MAP 160K by Uniqema.

3) Mention may be made, as carboxylates, of:
amido ether carboxylates (AEC), such as sodium lauryl amido ether carboxylate (3 EO), sold under the name Akypo Foam 30® by Kao Chemicals;
polyoxyethylenated carboxylic acid salts, such as oxyethylenated (6 EO) sodium lauryl ether carboxylate (65/25/10 C₁₂-C₁₄-C₁₆), sold under the name Akypo Soft 45 NV® by Kao Chemicals, polyoxyethylenated and carboxymethylated fatty acids originating from olive oil, sold under the name Olivem 400® by Biologia E Tecnologia, or oxyethylenated (6 EO) sodium tridecyl ether carboxylate, sold under the name Nikkol ECTD-6NEX® by Nikkol; and
salts of fatty acids (soaps) having a C₆ to C₂₂ alkyl chain which are neutralized with an organic or inorganic base, such as potassium hydroxide, sodium hydroxide, triethanolamine, N-methylglucamine, lysine and arginine.

4) Mention may in particular be made, as amino acid derivatives, of alkali salts of amino acids, such as:
sarcosinates, such as sodium lauroyl sarcosinate, sold under the name Sarkosyl NL 97® by Ciba or sold under the name Oramix L 30® by Seppic, sodium myristoyl sarcosinate, sold under the name Nikkol Sarcosinate MN® by Nikkol, or sodium palmitoyl sarcosinate, sold under the name Nikkol Sarcosinate PN® by Nikkol;
alaninates, such as sodium N-lauroyl-N-methylamidopropionate, sold under the name Sodium Nikkol Alaninate LN 30® by Nikkol or sold under the name Alanone ALE® by Kawaken, or triethanolamine N-lauroyl-N-methylalanine, sold under the name Alanone ALTA® by Kawaken;
glutamates, such as triethanolamine monococoyl glutamate, sold under the name Acylglutamate CT-12® by Ajinomoto, triethanolamine lauroyl glutamate, sold under the name Acylglutamate LT-12® by Ajinomoto;
aspartates, such as the mixture of triethanolamine N-lauroyl aspartate and triethanolamine N-myristoyl aspartate, sold under the name Asparack® by Mitsubishi;
glycine derivatives (glycinates), such as sodium N-cocoyl glycinate, sold under the names Amilite GCS-12® and Amilite GCK 12 by Ajinomoto;
citrates, such as the citric monoester of oxyethylenated (9 mol) coco alcohols, sold under the name Witconol EC 1129 by Goldschmidt; and
galacturonates, such as sodium dodecyl D-galactoside uronate, sold by Soliance.

5) Mention may be made, as sulphosuccinates, for example, of oxyethylenated (3 EO) lauryl (70/30 C₁₂/C₁₄) alcohol monosulphosuccinate, sold under the names Setacin 103 Special® and Rewopol SB-FA 30 K 4® by Witco, the disodium salt of a hemisulphosuccinate of C₁₂-C₁₄ alcohols, sold under the name Setacin F Special Paste® by Zschimmer Schwarz, oxyethylenated (2 EO) disodium oleamidosulphosuccinate, sold under the name Standapol SH 135® by Cognis, oxyethylenated (5 EO) lauramide monosulphosuccinate, sold under the name Lebon A-5000® by Sanyo, the disodium salt of oxyethylenated (10 EO) lauryl citrate monosulphosuccinate, sold under the name Rewopol SB CS 50® by Witco, or ricinoleic monoethanolamide monosulphosuccinate, sold under the name Rewoderm S 1333® by Witco. Use may also be made of polydimethylsiloxane sulphosuccinates, such as disodium PEG-12 dimethicone sulphosuccinate, sold under the name Mackanate-DC 30 by MacIntyre.

6) Mention may be made, as alkyl sulphates, for example, of triethanolamine lauryl sulphate (CTFA name: TEA lauryl sulphate), such as the product sold by Huntsman under the name Empicol TL40 FL or the product sold by Cognis under the name Texapon T42, which products are at 40% in aqueous solution. Mention may also be made of ammonium lauryl sulphate (CTFA name: ammonium lauryl sulphate), such as the product sold by Huntsman under the name Empicol AL 30FL, which is at 30% in aqueous solution.

7) Mention may be made, as alkyl ether sulphates, for example, of sodium lauryl ether sulphate (CTFA name: sodium laureth sulphate), such as that sold under the names Texapon N40 and Texapon AOS 225 UP by Cognis, or ammonium lauryl ether sulphate (CTFA name: ammonium laureth sulphate), such as that sold under the name Standapol EA-2 by Cognis.

8) Mention may be made, as sulphonates, for example, of α-olefinsulphonates, such as sodium α-olefinsulphonate (C₁₄-C₁₆), sold under the name Bio-Terge AS-40® by Stepan, sold under the names Witconate AOS Protégé® and Sulframine AOS PH 12® by Witco or sold under the name Bio-Terge AS-40 CG® by Stepan, secondary sodium olefinsulphonate, sold under the name Hostapur SAS 30® by Clariant; or linear alkylarylsulphonates, such as sodium xylenesulphonate, sold under the names Manrosol SXS30@, Manrosol SXS40® and Manrosol SXS93® by Manro.

9) Mention may be made, as isethionates, of acylisethionates, such as sodium cocoylisethionate, such as the product sold under the name Jordapon CI P® by Jordan.

10) Mention may be made, as taurates, of the sodium salt of palm kernel oil methyltaurate, sold under the name Hostapon CT Pat6@ by Clariant; N-acyl-N-methyltaurates, such as sodium N-cocoyl-N-methyltaurate, sold under the name Hostapon LT-SF® by Clariant or sold under the name Nikkol CMT-30-T® by Nikkol, sodium palmitoyl methyltaurate, sold under the name Nikkol PMT® by Nikkol, or sodium steraroyl methyltaurate, sold under the name Sunsoft 0-30S by Taiyo Kagaku.

11) The anionic derivatives of alkyl polyglucosides can in particular be citrates, tartrates, sulphosuccinates, carbonates and glycerol ethers obtained from alkyl polyglucosides.

Mention may be made, for example, of the sodium salt of cocoylpolyglucoside (1,4) tartaric ester, sold under the name Eucarol AGE-ET® by Cesalpinia, the disodium salt of cocoylpolyglucoside (1,4) sulphosuccinic ester, sold under the name Essai 512 MP® by Seppic, or the sodium salt of cocoylpolyglucoside (1,4) citric ester, sold under the name Eucarol AGE-EC® by Cesalpinia.

It is preferable that the (f) anionic surfactant be selected from taurate or glutamate, more preferably N-acyl-N-methyltaurate, and even more preferably sodium N-stearoyl-N-methyl-taurate.

It is preferable that the amino acid derivatives be acyl glycine derivatives or glycine derivatives, in particular acyl glycine salt.

The acyl glycine derivatives or glycine derivatives can be chosen from acyl glycine salts (or acyl glycinates) or glycine salts (or glycinates), and in particular from the following.

i) Acyl glycinates of formula (I):

R—HNCH₂COOX (I)
in which
R represents an acyl group R′C═O, with R′, which represents a saturated or unsaturated, linear or branched, hydrocarbon chain, preferably comprising from 10 to carbon atoms, preferably from 12 to 22 carbon atoms, more preferably from 14 to 22 carbon atoms and better still from 16 to 20 carbon atoms, and
X represents a cation chosen, for example, from the ions of alkali metals, such as Na, Li or K, preferably Na or K, the ions of alkaline earth metals, such as Mg, ammonium groups and their mixtures.

The acyl group can in particular be chosen from the lauroyl, myristoyl, behenoyl, palmitoyl, stearoyl, isostearoyl, olivoyl, cocoyl or oleoyl groups and their mixtures.

Preferably, R is a cocoyl group.

ii) Glycinates of the following formula (II):

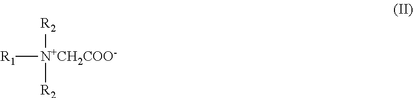

in which:
R₁ represents a saturated or unsaturated, linear or branched, hydrocarbon chain comprising from 10 to 30 carbon atoms, preferably from 12 to 22 carbon atoms and better still from 16 to 20 carbon atoms; R₁ is advantageously chosen from the lauryl, myristyl, palmityl, stearyl, cetyl, cetearyl or oleyl groups and their mixtures and preferably from the stearyl and oleyl groups,
the R₂ groups, which are identical or different, represent an R″OH group, R″ being an alkyl group comprising from 2 to 10 carbon atoms, preferably from 2 to 5 carbon atoms.

Mention may be made, as the compound of formula (I), for example, of the compounds carrying the INCI name sodium cocoyl glycinate, such as, for example, Amilite GCS-12, sold by Ajinomoto, or potassium cocoyl glycinate, such as, for example, Amilite GCK-12 from Ajinomoto.

Use may be made, as compounds of formula (II), of dihydroxyethyl oleyl glycinate or dihydroxyethyl stearyl glycinate.

The amount of the (f) anionic surfactant may range from 0.01 to 20% by weight, preferably from 0.05 to 10% by weight, and more preferably from 0.1 to 5% by weight, relative to the total weight of the composition.

(Additional Surfactant)

The composition according to the present invention may further comprise at least one nonionic surfactant different from the above ingredient (b) and/or at least one additional ionic surfactant different from the above ingredient (f). A single type of additional surfactant may be used, but two or more different types of additional surfactant may be used in combination.

As the additional surfactant, at least one nonionic surfactant with an HLB value less than 8.0 or more than 14 may be used.

As the additional nonionic surfactant, mention may be made of those listed for the above ingredient (b) except that the additional nonionic surfactant has an HLB value of less than 8.0, preferably less than 9.0, and more preferably less than 10.0, and more than 14, preferably more than 13.5, and more preferably more than 13.0.

As the additional surfactant(s), cationic surfactants and/or amphoteric surfactants may be used.

(Cationic Surfactant)

The cationic surfactant is not limited. The cationic surfactant may be selected from the group consisting of optionally polyoxyalkylenated, primary, secondary or tertiary fatty amine salts, quaternary ammonium salts, and mixtures thereof.

Examples of quaternary ammonium salts that may be mentioned include, but are not limited to:

those of general formula (I) below:

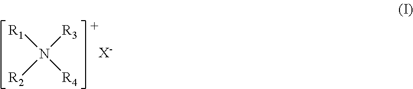

wherein

R₁, R₂, R₃, and R₄, which may be identical or different, are chosen from linear and branched aliphatic radicals comprising from 1 to 30 carbon atoms and optionally comprising heteroatoms such as oxygen, nitrogen, sulfur and halogens. The aliphatic radicals may be chosen, for example, from alkyl, alkoxy, C₂-C₆ polyoxyalkylene, alkylamide, (C₁₂-C₂₂)alkylamido(C₂-C₆)alkyl, (C₁₂-C₂₂)alkylacetate and hydroxyalkyl radicals; and aromatic radicals such as aryl and alkylaryl; and X⁻ is chosen from halides, phosphates, acetates, lactates, (C₂-C₆) alkyl sulfates and alkyl- or alkylaryl-sulfonates; quaternary ammonium salts of imidazoline;

diquaternary ammonium salts; and

quaternary ammonium salts comprising at least one ester function.

The quaternary ammonium salts mentioned above that may be used in compositions according to the invention include, but are not limited to, tetraalkylammonium chlorides, for instance dialkyldimethylammonium and alkyltrimethylammonium chlorides in which the alkyl radical comprises from about 12 to 22 carbon atoms, such as behenyltrimethylammonium, distearyldimethylammonium, cetyltrimethylammonium and benzyldimethylstearylammonium chloride; palmitylamidopropyltrimethylammonium chloride; and stearamidopropyldimethyl(myristyl acetate)ammonium chloride, sold under the name “Ceraphyl® 70” by the company Van Dyk.

According to one embodiment, the cationic surfactant that may be used in the compositions of the invention is chosen from quaternary ammonium salts, for example from behenyltrimethylammonium chloride, cetyltrimethylammonium chloride, Quaternium-83, Quaternium-87, Quaternium-22, behenylamidopropyl-2,3-dihydroxypropyldimethylammonium chloride, palmitylamidopropyltrimethylammonium chloride, and stearamidopropyldimethylamine.

(Amphoteric Surfactant)

The amphoteric surfactant is not limited. The amphoteric or zwitterionic surfactants can be, for example (nonlimiting list), amine derivatives such as aliphatic secondary or tertiary amine, and optionally quaternized amine derivatives, in which the aliphatic radical is a linear or branched chain comprising 8 to 22 carbon atoms and containing at least one water-solubilizing anionic group (for example, carboxylate, sulphonate, sulphate, phosphate or phosphonate).

Among the amidoaminecarboxylated derivatives, mention may be made of the products sold under the name Miranol, as described in U.S. Pat. Nos. 2,528,378 and 2,781,354 and classified in the CTFA dictionary, 3rd edition, 1982 (the disclosures of which are incorporated herein by reference), under the names Amphocarboxyglycinates and Amphocarboxypropionates, with the respective structures:

R₁—CONHCH₂CH₂—N⁺(R₂)(R₃)(CH₂COO⁻)

in which:

R₁ denotes an alkyl radical of an acid R₁—COOH present in hydrolysed coconut oil, a heptyl, nonyl or undecyl radical,

R₂ denotes a beta-hydroxyethyl group, and

R₃ denotes a carboxymethyl group; and

R₁′—CONHCH₂CH₂—N(B)(C)

in which:

B represents —CH₂CH₂OX′,

C represents —(CH₂)_{z}—Y′, with z=1 or 2,

X′ denotes a —CH₂CH₂—COOH group, —CH₂—COOZ′, —CH₂CH₂—COOH, —CH₂CH₂—COOZ′ or a hydrogen atom,

Y′ denotes —COOH, —COOZ′, —CH₂—CHOH—SO₃Z′ or a —CH₂—CHOH—SO₃H radical,

Z′ represents an ion of an alkaline or alkaline earth metal such as sodium, an ammonium ion or an ion issued from an organic amine, and

R₁′ denotes an alkyl radical of an acid R₁′—COOH present in coconut oil or in hydrolysed linseed oil, an alkyl radical, such as a C₇, C₉, C₁₁ or C₁₃ alkyl radical, a C₁₇ alkyl radical and its iso form, or an unsaturated C₁₇ radical.

It is preferable that the amphoteric surfactant be selected from (C₈-C₂₄)-alkyl amphomonoacetates, (C₈-C₂₄)alkyl amphodiacetates, (C₈-C₂₄)alkyl amphomonopropionates, and (C₈-C₂₄)alkyl amphodipropionates.

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphopropionate, Disodium Caprylamphodipropionate, Disodium Caprylamphodipropionate, Lauroamphodipropionic acid and Cocoamphodipropionic acid.

By way of example, mention may be made of the cocoamphodiacetate sold under the trade name Miranol® C2M concentrate by the company Rhodia Chimie.

Preferably, the amphoteric surfactant may be a betaine.

The betaine-type amphoteric surfactant is preferably selected from the group consisting of alkylbetaines, alkylamidoalkylbetaines, alkylsulfobetaines, alkylphosphobetaines, and alkylamidoalkylsulfobetaines, in particular, (C₈-C₂₄)alkylbetaines, (C₈-C₂₄)alkylamido(C₁-C₈)alkylbetaines, (C₈-C₂₄)alkylsulphobetaines, and (C₈-C₂₄)alkylamido(C₁-C₈)alkylsulphobetaines. In one embodiment, the amphoteric surfactants of betaine type are chosen from (C₈-C₂₄)alkylbetaines, (C₈-C₂₄)alkylamido(C₁-C₈)alkylsulphobetaines, (C₈-C₂₄)alkylsulphobetaines, and alkyl(C₈-C₂₄)phosphobetaines.

Non-limiting examples that may be mentioned include the compounds classified in the CTFA dictionary, 9th edition, 2002, under the names cocobetaine, laurylbetaine, cetylbetaine, coco/oleamidopropylbetaine, cocamido propyl betaine, palmitamido propylbetaine, stearamidopropylbetaine, cocamidoethylbetaine, cocamidopropylhydroxysultaine, oleamidopropylhydroxysultaine, cocohydroxysultaine, laurylhydroxysultaine, and cocosultaine, alone or as mixtures.

The betaine-type amphoteric surfactant is preferably an alkylbetaine and an alkylamidoalkylbetaine, in particular cocobetaine and cocamidopropylbetaine.

The amount of the additional surfactant(s) may be from 0.01 to 20% by weight, preferably from 0.10 to 10% by weight, and more preferably from 1 to 5% by weight, relative to the total weight of the composition.

(Polyol)

The composition according to the present invention may further comprise at least one polyol. A single type of polyol may be used, but two or more different types of polyol may be used in combination.

The term “polyol” here means an alcohol having two or more hydroxy groups, and does not encompass a saccharide or a derivative thereof. The derivative of a saccharide includes a sugar alcohol which is obtained by reducing one or more carbonyl groups of a saccharide, as well as a saccharide or a sugar alcohol in which the hydrogen atom or atoms in one or more hydroxy groups thereof has or have been replaced with at least one substituent such as an alkyl group, a hydroxyalkyl group, an alkoxy group, an acyl group or a carbonyl group.

The polyol may be a C₂-C₁₂ polyol, preferably a C₂-C₉ polyol, comprising at least 2 hydroxy groups, and preferably 2 to 5 hydroxy groups.

The polyol may be a natural or synthetic polyol. The polyol may have a linear, branched or cyclic molecular structure.

The polyol may be selected from glycerins and derivatives thereof, and glycols and derivatives thereof. The polyol may be selected from the group consisting of glycerin, diglycerin, polyglycerin, ethyleneglycol, propyleneglycol, dipropyleneglycol, butyleneglycol, pentyleneglycol, hexyleneglycol, 1,3-propanediol, 1,5-pentanediol, caprylyl glycol, polyethyleneglycol (5 to 50 ethyleneoxide groups), and sugars such as sorbitol.

The polyol may be present in an amount ranging from 0.01 to 30% by weight, and preferably 50 from 0.1 to 25% by weight, such as from 1 to 20% by weight, relative to the total weight of the composition.

(Thickening Agent)

The composition according to the present invention may further comprise at least one thickening agent. A single type of thickening agent may be used, but two or more different types of thickening agent may be used in combination.

The thickening agent may be selected from organic and inorganic thickeners.

The organic thickeners may be chosen from at least one of:
(i) associative thickeners;
(ii) crosslinked acrylic acid homopolymers;
(iii) crosslinked copolymers of (meth)acrylic acid and of (C₁-C₆)alkyl acrylate;
(iv) nonionic homopolymers and copolymers comprising at least one of ethylenically unsaturated ester monomers and ethylenically unsaturated amide monomers;
(v) ammonium acrylate homopolymers and copolymers of ammonium acrylate and of acrylamide;
(vi) polysaccharides; and
(vii) C₁₂-C₃₀ fatty alcohols.

The thickening agent is preferably selected from associative thickeners and polysaccharides such as starch and xanthan gum.

As used herein, the expression “associative thickener” means an amphiphilic thickener comprising both hydrophilic units and hydrophobic units, for example, comprising at least one C₈-C₃₀ fatty chain and at least one hydrophilic unit.

The viscosity of the composition according to the present invention is not particularly limited. The viscosity can be measured at 25° C. with viscosimeters or rheometers preferably with coneplan geometry. Preferably, the viscosity of the composition according to the present invention can range, for example, from 1 to 2000 Pa·s, and preferably from 1 to 1000 Pa·s at 25° C. and 1 s⁻¹.

The thickening agent may be present in an amount ranging from 0.001 to 10% by weight, and preferably from 0.01 to 10% by weight, such as from 0.1 to 5% by weight, relative to the total weight of the composition.

(Other Optional Ingredients)

The composition according to the present invention may also comprise an effective amount of other optional ingredients, such as various common adjuvants, antiageing agents, whitening agents, anti-greasy skin agents, sequestering agents such as EDTA and etidronic acid, UV screening agents, preserving agents, vitamins or provitamins, for instance, panthenol, opacifiers, fragrances, plant extracts, cationic polymers and so on.

For example, the composition according to the present invention may further comprise at least one organic solvent. Thus, the organic solvent is preferably water miscible. As the organic solvent, there may be mentioned, for example, C₁-C₄ alkanols, such as ethanol and isopropanol; aromatic alcohols such as benzyl alcohol and phenoxyethanol; analogous products; and mixtures thereof.

The organic water-miscible solvents may be present in an amount ranging from less than 10% by weight, preferably from 5% by weight or less, and more preferably from 1% by weight or less, relative to the total weight of the composition.

[Preparation and Properties]

The composition according to the present invention can be prepared by mixing the above essential and optional ingredients in accordance with a conventional process. The conventional process includes mixing with a high pressure homogenizer (a high energy process). Alternatively, the composition can be prepared by a low energy process such as phase inversion temperature process (PIT), phase inversion concentration (PIC), autoemulsification, and the like. Preferably, the composition is prepared by a low energy process.

The composition according to the present invention is in the form of a nano- or micro-emulsion.

A “micro-emulsion” may be defined in two ways, namely, in a broader sense and in a narrower sense. That is to say, there is the one case (“microemulsion in the narrow sense”) in which the microemulsion refers to a thermodynamically stable isotropic single liquid phase containing a ternary system having three ingredients of an oily component, an aqueous component and a surfactant, and there is the other case (“micro-emulsion in the broad sense”) in which among thermodynamically unstable typical emulsion systems the microemulsion additionally includes those such emulsions presenting transparent or translucent appearances due to their smaller particle sizes (Satoshi Tomomasa, et al., OilChemistry, Vol. 37, No. 11 (1988), pp. 48-53). The “micro-emulsion” as used herein refers to a “micro-emulsion in the narrow sense”, i.e., a thermodynamically stable isotropic single liquid phase.

The micro-emulsion refers to either one state of an O/W (oil-in-water) type microemulsion in which oil is solubilized by micelles, a W/O (water-in-oil) type microemulsion in which water is solubilized by reverse micelles, or a bicontinuous microemulsion in which the number of associations of surfactant molecules is rendered infinite so that both the aqueous phase and oil phase have a continuous structure.

The micro-emulsion may have a dispersed phase with a number average diameter of 100 nm or less, preferably 50 nm or less, and more preferably 20 nm or less, measured by laser granulometry.

The “nano-emulsion” here means an emulsion characterized by a dispersed phase with a size of less than 350 nm, the dispersed phase being stabilized by a crown of the (b) nonionic surfactant that may optionally form a liquid crystal phase of lamellar type, at the dispersed phase/continuous phase interface. In the absence of specific opacifiers, the transparency of the nano-emulsions arises from the small size of the dispersed phase, this small size being obtained by virtue of the use of mechanical energy and especially a high-pressure homogenizer.

Nano-emulsions can be distinguished from microemulsions by their structure. Specifically, 50 micro-emulsions are thermodynamically stable dispersions formed from, for example, the (b) nonionic surfactant micells swollen with the (a) oil. Furthermore, microemulsions do not require substantial mechanical energy in order to be prepared.

The nano-emulsion may have a dispersed phase with a number average diameter of 300 nm or less, preferably 200 nm or less, and more preferably 100 nm or less, measured by laser granulometry.

The composition according to the present invention may be in the form of an O/W nano- or micro-emulsion, a W/O nano- or micro-emulsion or a bicontinuous emulsion. It is preferable that the composition according to the present invention be in the form of an O/W nano- or micro-emulsion.

It is preferable that the composition according to the present invention be in the form of an O/W emulsion. The (a) oil may be in the form of droplets with a number average particle size of, preferably 300 nm or less, more preferably 200 nm or less, and more preferably from 100 nm or less.

The composition according to the present invention can have a transparent or slightly translucent appearance, preferably a transparent appearance.

The transparency may be determined by measuring the turbidity with, for example, a 2100Q (HACH) with a round cell (25 mm in diameter X 60 mm height) and a tungsten filament lamp.

The composition according to the present invention may have a turbidity of less than 300, preferably 200 or less, more preferably 100 or less, and even more preferably 46.0 or less.

[Process and Use]

The composition according to the present invention can be used for a non-therapeutic process, such as a cosmetic process, for treating a keratin substance such as the skin, the hair, mucous membranes, the nails, the eyelashes, the eyebrows and the scalp, comprising the step of applying the composition according to the preseht invention to the keratin substance.

Thus, in one particular embodiment, the composition according to the present invention is a cosmetic composition.

The present invention also relates to a use of the composition according to the present invention as it is or in care products and/or washing products and/or make-up products and/or make-up-removing products, for body and/or facial skin and/or mucous membranes and/or the scalp and/or the hair and/or the nails and/or the eyelashes and/or the eyebrows.

In other words, the composition according to the present invention can be used, as it is, as the above product. Alternatively, the composition according to the present invention can be used as an element of the above product. For example the composition according to the present invention can be added to or combined with any other elements to form the above product.

The care product may be a lotion, a cream, a serum, a hair tonic, a hair conditioner, a sun 50 screening agent, and the like. The washing product may be a shampoo, a face wash, a hand wash and the like. The make-up product may be a foundation, a mascara, a lipstick, a lip gloss, a blusher, an eye shadow, a nail varnish, and the like. The make-up-removing product may be a make-up cleansing agent and the like.

The present invention also relates to the use of (d) at least one compound selected from Vitamin B3 and derivatives thereof, as explained above, for enhancing or improving the penetration of (c) at least one ceramide compound, as explained above, into the keratin substance, wherein the (c) ceramide compound is present in a nano- or micro-emulsion, comprising:
(a) at least one oil;
(b) at least one nonionic surfactant with an HLB value of from 8.0 to 14.0, preferably from 9.0 to 13.5, and more preferably from 10.0 to 13.0; and
(e) water.

### EXAMPLES

The present invention will be described in a more detailed manner by way of examples. However, these examples should not be construed as limiting the scope of the present invention.

### Example 1 and Comparative Examples 1 and 2

The following compositions according to Example (Ex.) 1 and Comparative Examples (Comp. Ex.) 1 and 2, shown in Table 1, were prepared by mixing the components shown in Table 1. The numerical values for the amounts of the components shown in Table 1 are all based on “% by weight” as active raw materials.

TABLE 1

| | | Comp. | Comp. |
|---|---|---|---|
| | Ex. 1 | Ex. 1 | Ex. 2 |
| 2-Oleamido-1,3-Octanediol*¹ | 0.2 | 0.2 | 0.2 |
| Citric Acid | 0.01 | 0.01 | 0.01 |
| Octyldodecanol | 0.8 | 0.8 | 0.8 |
| Ethylhexyl Palmitate | 3 | 3 | 3 |
| Squalane | 0.2 | 0.2 | 0.2 |
| Xanthan Gum | 0.1 | 0.1 | 0.1 |
| Phenoxyethanol | 0.5 | 0.5 | 0.5 |
| Chlorphenesin | 0.3 | 0.3 | 0.3 |
| Butylene Glycol | 10 | 10 | 10 |
| Water | qsp 100 | qsp 100 | qsp 100 |
| Glycerin | 3 | 3 | 3 |
| Propylene Glycol | 7 | 7 | 7 |
| Caprylyl Glycol | 0.3 | 0.3 | 0.3 |
| PPG-6 Decyl Tetradeceth-30 | 1 | 1 | 4.2 |
| Sodium Methyl Stearoyl Taurate | 0.2 | 0.2 | 0.2 |
| Polyglyceryl-5 Laurate | 4 | 4 | — |
| Niacinamide | 0.75 | — | 0.75 |
| Penetration Amount of Active Ingredient | 123.0 | 58.2 | 69.9 |
| Turbidity | 45.5 | 47 | 934 |

*¹Ceramide

[Evaluations]

(Penetration Amount of Active Ingredient)

The penetration amount of the ceramide (2-oleamido-1,3-octanediol) in each of the compositions according to Example 1 and Comparative Examples 1 and 2 was determined by using a flank pig skin. The experimental procedure is described below in a detailed manner.

Experimental Procedure:
A receptor fluid (RF) was prepared by mixing 4 g of Tween 80, 10 g of phosphate buffered saline, and 86 g of water in a beaker and stirring until it became isotropic. The obtained RF had a pH value of 7.4.
A static Franz Cell with exposure area of 2 cm² and receptor compartment of 3 ml was used.
A stirrer was put in a Frantz cell and the receptor compartment was filled with RF.
Full thickness frozen pig flank skin was punched at 32 mm diameter. Skin thickness should be lower than 2.5 mm.
Water bath was adjusted to obtained skin temperature at 32° C.±1° C.
A well-controlled amount of formulation (i.e. 5 mg/cm²) was applied on the skin surface.
After 16 hours, the skin surface was washed with an appropriate protocol to guarantee that all chemical remaining at the skin surface was removed.
Stratum Corneum was removed by 30 consecutive tape strips (DSquam), and extracted with Methanol (10 ml). Methanol extract was filtered on filter millex HV 0.45 m before LC/MS/MS analysis.
Remaining epidermis and dermis was extracted with Methanol (5 ml). Methanol extract was filtered on filter millex HV 0.45 m before LC/MS/MS analysis.
For each formulation, three donors in triplicate (n=9) were used.

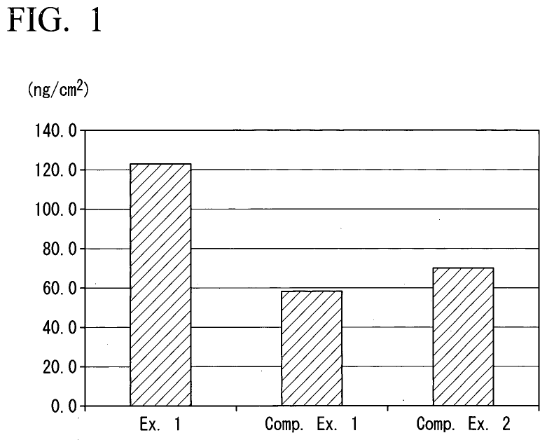

The mean value of the amount (ng per cm² of membrane) of the ceramide which passed through the flank pig skin is shown in Table 1, as well as FIG. 1.

(Turbidity)

The turbidity of the compositions according to Example 1 and Comparative Examples 1 and 2 were measured with a 2100Q (HACH) with a round cell (25 mm in diameter X 60 mm height) and a tungsten filament lamp.

The results are shown in Table 1.

It is recognized by comparing Example 1 and Comparative Example 1 that the composition according to Example 1 can provide enhanced or improved penetration of ceramide (2-oleamido-1,3-octanediol) through the epidermis and dermis, due to Vitamin B3 (niacinamide), as compared to the composition according to Comparative Example 1.

It is also recognized by comparing Example 1 and Comparative Example 2 that the composition according to Example 1 can provide enhanced or improved penetration of ceramide (2-oleamido-1,3-octanediol) through the epidermis and dermis, as compared to the composition according to Comparative Example 2, because the composition according to Example 1 is in the form of a nano- or micro-emulsion, while the composition according to Comparative Example 2 is not in the form of a nano- or micro-emulsion.

1.-15. (canceled)

16. A composition in the form of a nano- or micro-emulsion, comprising:
at least one oil;
at least one nonionic surfactant having an HLB value ranging from 8.0 to 14.0;
at least one ceramide compound;
at least one compound selected from Vitamin B3 or derivatives thereof; and
water.

17. The composition according to claim 16, wherein the at least one oil is chosen from ester oils, hydrocarbon oils, fatty alcohols, or mixtures thereof.

18. The composition according to claim 16, wherein the at least one oil is present in an amount ranging from 0.1 to 30% by weight, relative to the total weight of the composition.

19. The composition according to claim 16, wherein the at least one oil is present in an amount ranging from 0.5 to 20% by weight, relative to the total weight of the composition.

20. The composition according to claim 16, wherein the at least one nonionic surfactant is chosen from:
surfactants that are fluid at a temperature of less than or equal to 45° C., chosen from the esters of at least one polyol and at least one fatty acid, wherein:
the at least one polyol is chosen from the group formed by polyethylene glycol comprising from 1 to 60 ethylene oxide units, sorbitan, glycerol comprising from 2 to 30 ethylene oxide units, or polyglycerols comprising from 2 to 12 glycerol units; and
the at least one fatty acid comprises at least one saturated or unsaturated, linear or branched C₈-C₂₂ alkyl chain;
mixed esters of fatty acid or of fatty alcohol, of carboxylic acid and of glycerol;
fatty acid esters of sugars and fatty alcohol ethers of sugars;
surfactants that are solid at a temperature of less than or equal to 45° C., chosen from fatty esters of glycerol, fatty esters of sorbitan and oxyethylenated fatty esters of sorbitan, ethoxylated fatty ethers, or ethoxylated fatty esters;
block copolymers of ethylene oxide and of propylene oxide;
polyoxyethylenated (1-40 EO) and polyoxypropylenated (1-30 PO) alkyl (C₁₆-C₃₀) ethers; or
silicone surfactants.

21. The composition according to claim 16, wherein the at least one nonionic surfactant is chosen from:
polyethylene glycol isostearate or oleate (8 to 10 mol of ethylene oxide);
polyethylene glycol isocetyl, behenyl ether or isostearyl ether (8 to 10 mol of ethylene oxide);
polyglyceryl monolaurate or dilaurate comprising 3 to 6 glycerol units;
polyglyceryl mono(iso)stearate comprising 3 to 6 glycerol units;
polyglyceryl monooleate comprising 3 to 6 glycerol units; or
polyglyceryl dioleate comprising 3 to 6 glycerol units.

22. The composition according to claim 21 wherein the at least one nonionic surfactant is chosen from polyglyceryl fatty acid esters.

23. The composition according to claim 16, wherein the at least one nonionic surfactant is present in an amount ranging from 0.1 to 30% by weight, relative to the total weight of the composition.

24. The composition according to claim 16, wherein the at least one nonionic surfactant is present in an amount ranging from 0.5 to 20% by weight, relative to the total weight of the composition.

25. The composition according to claim 16, wherein the at least one ceramide compound is represented by formula (I): 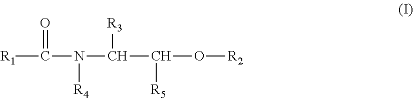
wherein:
R₁ denotes:
a saturated or unsaturated and linear or branched C₁-C₅₀ hydrocarbon radical, optionally substituted by one or more hydroxyl groups optionally esterified by an acid R₇COOH, R₇ being an optionally mono- or polyhydroxylated C₁-C₃₅ hydrocarbon radical in which one more hydroxyl is optionally esterified by an optionally mono- or polyhydroxylated C₁-C₃₅ fatty acid;
an R″—(NR—CO)—R′ radical in which R denotes a hydrogen atom or a mono- or polyhydroxylated C₁-C₂₀ hydrocarbon radical, wherein R′ and R″ are hydrocarbon radicals, the sum of the carbon atoms of which is between 9 and 30, and R′ being a divalent radical; or
an R₈—O—CO—(CH₂)ₚ radical in which R₈ denotes a C₁-C₂₀ hydrocarbon radical and p is an integer varying from 1 to 12;
R₂ is chosen from a hydrogen atom, a radical of saccharide type, a sulphate or phosphate residue, a phosphorylethylamine radical, or a phosphorylethylammonium radical;
R₃ denotes a hydrogen atom, a nonhydroxylated C₁-C₃₃ hydrocarbon radical, or a hydroxylated C₁-C₃₃ hydrocarbon radical, wherein:
one or more hydroxyl is optionally esterified by an inorganic acid or an acid R₇COOH, R₇ being an optionally mono- or polyhydroxylated C₁-C₃₅ hydrocarbon radical in which one more hydroxyl is optionally esterified by an optionally mono- or polyhydroxylated C₁-C₃₅ fatty acid; and
one or more hydroxyl is optionally etherified by a (glycosyl)ₙ, (galactosyl)ₘ, sulphogalactosyl, phosphorylethylamine or phosphorylethylammonium radical, wherein n is an integer ranging from 1 to 4 and m is an integer ranging from 1 to 8;
wherein R₃ is optionally substituted by one or more C₁-C₁₄ alkyl radical;
R₄ denotes a hydrogen atom, a methyl or ethyl radical, an optionally hydroxylated C₃-C₅₀ hydrocarbon radical, or a —CH₂—CHOH—CH₂—O—R₆ radical wherein R₆ denotes a C₁₀-C₂₆ hydrocarbon radical, or an R₈—O—CO—(CH₂)ₚ radical wherein R₈ denotes a C₁-C₂₀ hydrocarbon radical and p is an integer ranging from 1 to 12;
R₅ denotes a hydrogen atom or an optionally mono- or polyhydroxylated C₁-C₃₀ hydrocarbon radical wherein at least one hydroxyl is etherified by a (glycosyl)ₙ, (galactosyl)ₘ, sulphogalactosyl, phosphorylethylamine, or phosphorylethylammonium radical, wherein n is an integer ranging from 1 to 4 and m is an integer ranging from 1 to 8;
wherein if R₃ and R₅ denote hydrogen, or if R₃ denotes hydrogen and R₅ denotes methyl, then R₄ does not denote a hydrogen atom or a methyl or ethyl radical.

26. The composition according to claim 16, wherein the at least one ceramide compound is selected from 2-N-linoleoylaminooctadecane-1,3-diol, 2-N-oleoylaminooctadecane-1,3-diol, 2-N-palmitoylaminooctadecane-1,3-diol, 2-N-stearoylaminooctadecane-1,3-diol, 2-N-behenoylaminooctadecane-1,3-diol, 2-N-[2-hydroxypalmitoyl]aminooctadecane-1,3-diol, 2-N-stearoylaminooctadecane-1,3,4-triol, 2-N-palmitoylaminohexadecane-1,3-diol, or mixtures thereof.

27. The composition according to claim 16, wherein the at least one ceramide compound is present in an amount ranging from 0.01 to 20% by weight, relative to the total weight of the composition.

28. The composition according to claim 16, wherein the at least one ceramide compound is present in an amount ranging from 0.05 to 15% by weight, relative to the total weight of the composition.

29. The composition according to claim 16, wherein the at least one compound selected from Vitamin B3 or derivatives thereof has a log P of from −0.7 to 6.

30. The composition according to claim 16, wherein the at least one compound selected from Vitamin B3 or derivatives thereof is niacinamide.

31. The composition according to claim 16, wherein the at least one compound selected from Vitamin B3 or derivatives thereof is present in an amount ranging from 0.01 to 20% by weight, relative to the total weight of the composition.

32. The composition according to claim 16, wherein the at least one compound selected from Vitamin B3 or derivatives thereof is present in an amount ranging from 0.05 to 15% by weight, relative to the total weight of the composition.

33. The composition according to claim 16, wherein the composition further comprises at least one anionic surfactant.

34. The composition according to claim 16, wherein the at least one nonionic surfactant has an HLB value ranging from 9.0 to 13.5.

35. A cosmetic process for treating a keratin substance, the method comprising:
applying a composition to the keratin substance, wherein the composition comprises:
at least one oil;
at least one nonionic surfactant with an HLB value of from 8.0 to 14.0;
at least one ceramide compound;
at least one compound selected from Vitamin B3 or derivatives thereof; and
water.
